(19) United States
(12) Patent Application Publication
(10) Pub. No.: US 2025/0321221 A1
(43) Pub. Date: Oct. 16, 2025

(54) Headspace Eliminating Microtiter Plate Lid

(71) Applicant: Agilent Technologies, Inc., Santa Clara, CA (US)

(72) Inventors: Tom Erik Pedersen, Cork (IE); Ryan McGarrigle, Glanmire, Cork (IE); Conn Carey, Ballygarvan Cork (IE); Joseph Zakrzewski, Chicopee, MA (US)

(21) Appl. No.: 18/849,126

(22) PCT Filed: Apr. 7, 2023
(86) PCT No.: PCT/US2023/017808

§ 371 Date: Sep. 20, 2024

Related U.S. Application Data
(60) Provisional application No. 63/328,901, filed on Apr. 8, 2022.

(51) Int. Cl. G01N 33/50; B01L 3/00

(52) U.S. Cl. CPC G01N 33/5005; B01L 3/50853; B01L 2200/025; B01L 2200/141; B01L 2300/0829

(57) ABSTRACT

A microtiter plate assembly is disclosed that includes a base having a plurality of wells, a lid having a lid and a plurality of projections corresponding to the plurality of wells, a magnetic plate adapter including at least one magnet, and a force distribution plate that includes, or is coated with a magnetic or paramagnetic material. The magnetic plate adapter and the force distribution plate provide an amount of force necessary to reduce warping of large microtiter plate assays without increasing the footprint of the microtiter plate.

### RELATED APPLICATIONS

The present application is the national stage entry of International Patent Application No. PCT/US2023/017808 having a filing date of Apr. 7, 2023, which claims priority to U.S. Provisional Application Ser. No. 63/328,901, filed on Apr. 8, 2022, both of which are incorporated herein in their entirety by reference hereto.

### BACKGROUND

Biomedical analysis and imaging plays a role in a large number of diagnostic and therapeutic procedures including visualizing external and internal anatomical and physiological structures, features, and systems and evaluating complex biological events in the body at the organ, tissue, cellular, and molecular levels. For instance, biomedical imaging and analysis techniques are particularly well suited in assay devices. An assay is a qualitative and/or quantitative analysis of an unknown analyte. In one aspect, for instance, an assay device can be used to conduct an analysis of the type and concentration of an analyte contained in a cellular sample. These types of devices are well suited to analyzing living cells and providing useful information regarding the metabolic processes that are occurring inside the cells. For instance, the devices can provide real-time cell analyte measurements that provide a clear window into the critical functions driving cell processes, such as signaling, proliferation, activation, toxicity, and biosynthesis. More particularly, these devices can generate a metabolic phenotype and describe cellular microenvironment in a relatively short amount of time.

It is desirable to conduct these analyses using microtiter plates having 96 wells or more while maintaining high sensitivity, such as by using low sample volumes or very short distances between sensor and cells. However, existing solutions lack the structural stability required for use with standard microtiter plates and therefore suffer from warping, which can lead to inaccuracies and lack of both sensitivity and consistency in assay measurements. This problem is also generally observed to worsen over time.

Attempts have been made to stabilize large microtiter plates by applying weights or clamps to minimize movement and warping. However, these solutions are incompatible with existing plate readers and therefore fail to meet specifications set forth by the Society for Biomolecular Screening (referred to herein as “SBS compliance”) metrics in terms of size and weight. In addition, attempts have been made to utilize magnets, however, proposed solutions failed to provide adequate force, failed the weight requirements for plate readers, or interfered with measurements due to the size of the magnets.

In addition, existing products lack the consistent measurement height needed to maintain high sensitivity at low sample volume. For instance, in order to achieve high sensitivity, the projections or stoppers on a microtiter plate lid need to extend the same distance into the respective well without large variations amongst other wells and projections located on the same microtiter plate. However, attempts to solve this problem have failed to provide a solution that provides such low coefficients of variation for microtiter plates having a large number of wells (e.g., have failed to provide edge or center support to microtiter plates having 96 wells or more).

Therefore, while equipment has been proposed to limit warping and improve sensitivity and consistency in microtiter plates, a need still exists for a microtiter plate assembly that exhibits low warping even at large array sizes. It would also be a benefit to provide a microtiter plate assembly that has a defined distance between the stoppers/projections with a low coefficient of variation. It would be a further benefit to provide an assembly that is compatible with existing microtiter plates and plate readers.

### SUMMARY

The present disclosure is generally directed to a microtiter plate assembly that includes a base, a lid, a magnetic plate adapter, and a force distributing plate. Furthermore, the base includes a plurality of wells in at least one outer row and at least one inner row and the lid includes a plurality of projections corresponding with the plurality of wells. In addition, at least a portion of the force distributing plate, the magnetic plate adaptor, or both the magnetic plate adapter and force distributing plate is formed from a magnetic or paramagnetic material, or has a magnetic or paramagnetic material disposed thereon, or has a magnetic material attached thereto, where the at least one magnet provides a force sufficient to reduce warping of the lid, such that the assembly exhibits a percent warp, defined as a percent change in signal of an average fluorescence signal of the at least one outer row to an average fluorescence signal of the at least one inner row, of about 30% or less.

The present disclosure is also generally directed to a microtiter plate assembly that includes a base, a lid, a magnetic plate adapter and a force distributing plate, where the base includes a plurality of wells spaced apart such that each well is adjacent to a second well, the lid includes a plurality of projections spaced apart corresponding with the array of the plurality of wells such that each projection is adjacent to a second projection, and the a magnetic plate adapter includes at least one magnet. In addition, at least a portion of the force distributing plate, the magnetic plate adaptor, or both the magnetic plate adapter and force distributing plate is formed from a magnetic or paramagnetic material, or has a magnetic or paramagnetic material disposed thereon, or has a magnetic material attached thereto. Furthermore, the plurality of projections extend from a proximal end adjacent to the lid to a distal end; and a height clearance is defined between the distal end of each projection and a well base of the respective well, where a percent variation of the height clearance between the projection and the well and the adjacent second projection and second well is about 10% or less.

In one aspect, the force distributing plate, the magnetic plate adaptor, or both the magnetic plate adapter and force distributing plate provides at least about 4 kilograms*force (kgf) to the lid. Moreover, in an aspect the assembly exhibits a percent warp of about 15% or less. In another aspect, the at least one magnet is vertically magnetized

In yet a further aspect, at least one of the magnetic plate adapter and the force distributing plate has a longitudinal length that is equal to or less than a longitudinal length of the base. Additionally or alternatively, in an aspect, the base contains an apron, wherein the apron is configured to releasably contain the at least one magnet. Moreover, in one aspect, at least one of the magnetic plate adapter and the force distributing plate is formed of aluminum, steel, combinations thereof, or alloys or derivatives thereof. In another aspect, the microtiter plate assembly is configured to be compatible with a plate reader and/or is Society for Biomolecular Screening (SBS) compliant. In an aspect, at least one of the magnetic plate adapter and the force distributing plate has a thickness of about 12 mm or less. In yet a further aspect, at least one of the magnetic plate adapter and the force distributing plate has a weight of about 600 g or less.

Additionally or alternatively, in one aspect, at least one of the magnetic plate adapter and the force distributing plate contains one or more apertures. In a further aspect, the assembly includes 96 wells and 96 corresponding projections. In yet another aspect, each projection comprises a radial notching having a radial cross section of about 0.8 mm2 or greater. In one aspect, the distal end of each projection is canted at an angle of about 5° or more relative to a plane of the lid. Furthermore, in an aspect, at least a portion of the assembly is formed from an acrylate polymer, or wherein at least a portion of the assembly is coated with a polymer having an oxygen transmission rate of about 15 cm³/m²/24 hours at 23° C. and 0% relative humidity. In another aspect, at least one of the lid and the force distributing plate can include a plurality of spacers.

The present disclosure is also generally directed to a method of measuring an oxygen consumption rate utilizing the assembly of any one or more of the discussed aspects. The method includes placing a sample and an oxygen-sensitive phosphorescent probe into a well, contacting the sample with the corresponding projection, and measuring the oxygen consumption rate with a fluorescence plate reader.

In one aspect, the sample has a seeding density of less than 70,000 cells, and the measuring is conducted in 45 minutes or less. In a further aspect, the seeding density is conducted at or below confluence of a selected cell type. In yet another aspect, the sample volume is 100 μL or less, preferably wherein the sample volume is 70 μL or less.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1A illustrates one aspect of a microtiter plate assembly according to the present disclosure in an exploded view;

FIG. 1B illustrates the aspect of FIG. 1A in an assembled view;

FIG. 1C shows a cross-sectional view of the aspect of FIG. 1B;

FIG. 2A illustrates one aspect of a microtiter plate assembly according to the present disclosure;

FIG. 2B illustrates an aspect of a microtiter plate lid according to FIG. 2A;

FIG. 3A illustrates a bottom-up view of another aspect of a microtiter plate lid according to the present disclosure;

FIG. 3B illustrates the microtiter plate lid of FIG. 3A along cross-section B-B;

FIG. 3C illustrates a side perspective of the microtiter plate lid of FIG. 3A;

FIG. 3D illustrates a top-down view of the microtiter plate lid of FIG. 3A;

FIG. 3E illustrates a cross-section view of FIG. 3D along D-D;

FIG. 3F illustrates a perspective view of the microtiter plate lid of FIG. 3A;

FIG. 4A illustrates another aspect of a microtiter plate lid according to the present disclosure;

FIG. 4B illustrates a microtiter plate base corresponding to the microtiter plate lid of FIG. 4A;

FIG. 4C illustrates an aspect of a microtiter plate lid of FIG. 4A in releasable associate with the microtiter plate base of FIG. 4B;

FIG. 4D illustrates an aspect of a microtiter assembly according to the present disclosure;

FIG. 5A illustrates a further aspect of a microtiter plate assembly according to the present disclosure;

FIG. 5B is a zoomed in perspective of a distal end of a projection of FIG. 5A;

FIG. 5C is a zoomed in perspective view of a distal end of a projection according to the present disclosure;

FIG. 6 is a graph of warping over time of an assembly according to the present disclosure measured in relative fluorescence units according to Example 1;

FIG. 7 is a graph of percent warp over 90 minutes according to Example 2;

FIGS. 8A-8D illustrate the test methods and data of Example 4;

FIG. 9A illustrates another aspect of a microtiter plate assembly according to the present disclosure in an exploded view;

FIG. 9B illustrates a top view of the lid of the microtiter plate assembly of FIG. 9A;

FIG. 9C illustrates a perspective top view of a force distributing plate that can be used with the microtiter plate assembly of FIG. 9A; and

FIG. 10 is a graph showing a comparison of the coefficient of variation of oxygen depletion measurements in the wells of a lid of the microtiter plate assembly of FIGS. 9A and 9B utilizing spacers having various thicknesses compared to a lid with no spacers.

Repeat use of reference characters in the present specification and drawings is intended to represent the same or analogous features or elements of the present invention.

### DETAILED DESCRIPTION

### Definitions and Test Methods

Warping, or warp, as used herein, can be defined as movement of the pillars and/or protrusions in the y-direction. This results in an increased height clearance, which will be discussed in greater detail below, and thus results in the need for increased sample volume. If warping occurs during an oxygen depletion assay, for example, it will cause inconsistencies in sample volume across the microplate, thus the measurement of different levels of oxygen depletion that are not comparable across multiple sample wells or chambers.

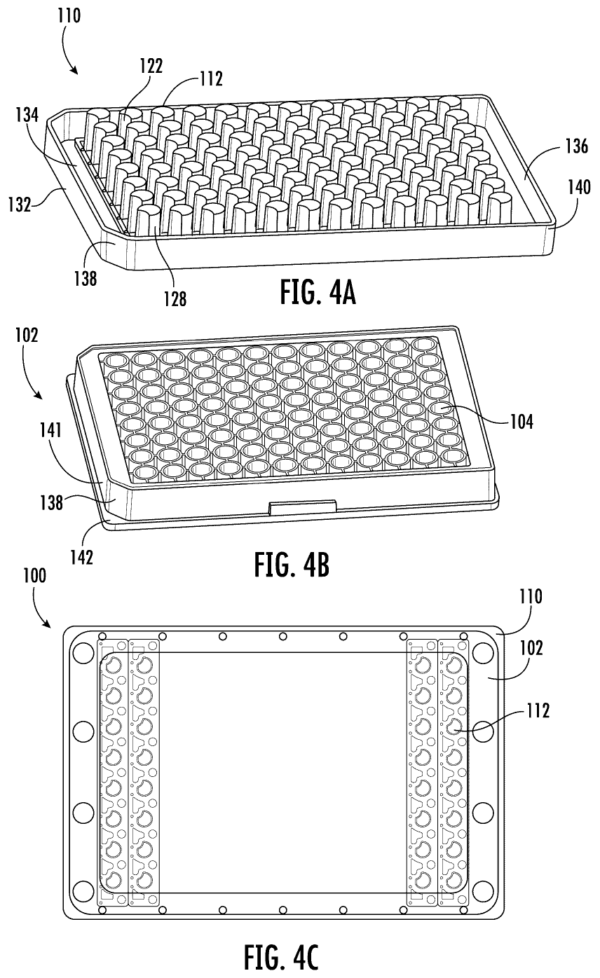

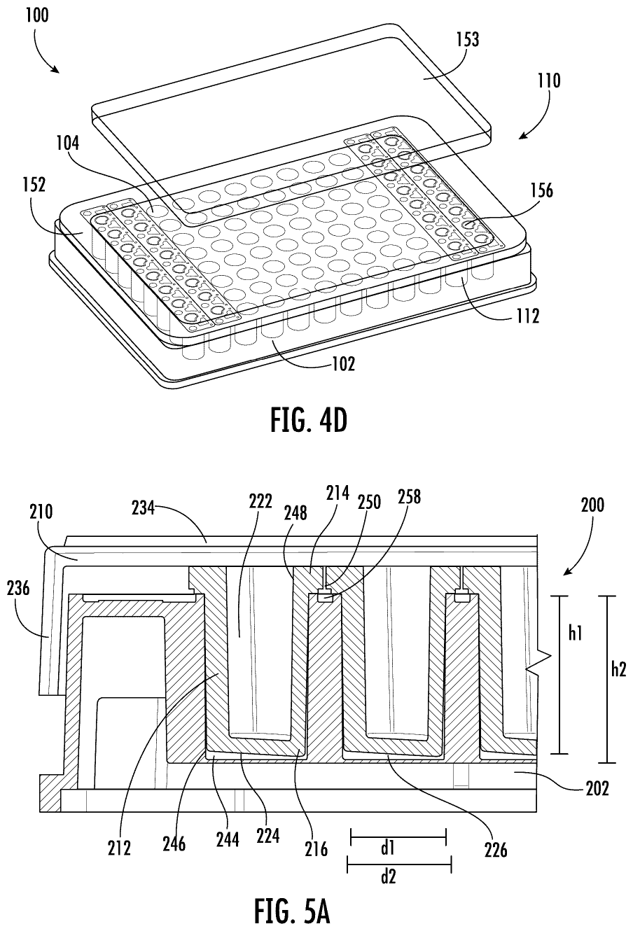

Furthermore, warping, or percent warp according to the present disclosure, and discussed in greater detail in example 1 below, can be measured by providing a 96 well plate, dissolving a phosphorescent reagent (e.g. MitoXpress Xtra, in one example), in a fluid sample contained in the wells, measuring a signal from the reagent, calculating an average fluorescence signal from each row of wells, and calculating a percent intensity of each row, expressed as a percent of the average signal of the middle rows (e.g. for a 96 well plate, there would be eight rows of twelve wells as shown generally in FIGS. 4A-4C, where rows three to six, or C, D, E, and F as shown in Example 1 and FIGS. 4A-4C, are inner rows, and rows one, two, seven, and eight, or A, B, G, and H, are outer rows). Namely, as will be discussed in greater detail below, this method assumes that increased fluorescence signal indicates increased sample volume when all other conditions (e.g., temperature, ambient oxygen) are kept constant.

As used herein, “translucence” or “translucent” means the light transmittance or optical density of a material which is in a range between transparent and opaque. Generally speaking, translucent materials allow light to pass through diffusely. A material that is translucent will allow a greater level of electromagnetic radiation in the visible spectrum to pass through it than an opaque or substantially opaque material but will allow a lower level of electromagnetic radiation in the visible spectrum to pass through it than a transparent or substantially transparent material. The translucent material can offer protection from phototoxicity while still allowing detection of the cells. A consequence is that cells cannot be observed from the top of the plate.

Oxygen concentration can be determined or indirectly interrogated by placing an oxygen-sensitive photoluminescent probe and a fluid test sample within a plurality of wells in a microtiter plate and ascertaining oxygen concentration within each well of the covered microtiter plate by exposing the oxygen-sensitive photoluminescent probe within each well to excitation radiation passed through the projection extending therein or the well bottom to create excited oxygen-sensitive photoluminescent material, measuring radiation emitted by the excited oxygen-sensitive photoluminescent material through the projection and the well bottom, and either converting the measured emission to a target-analyte concentration based upon a known conversion algorithm or measuring the probe/sensor signal in either intensity or lifetime modes. In one aspect, a suitable photoluminescent probe for such a measurement is MitoXpress® Xtra, available from Agilent Technologies. Instruments suitable for reading oxygen-sensitive photoluminescent probes within a cell sample are known and available from a number of sources, including the CLARIOstar plate reader from BMG Labtech GmbH of Ortenberg, Germany and the Synergy HTX from BioTek, an Agilent Technologies company. However, the photoluminescent material can also include an indicator dye incorporated in an oxygen permeable polymeric matrix, and it should be understood that electrochemical sensors can also be used to determine oxygen consumption.

Oxygen Consumption Rate (OCR), which can also be referred to as oxygen depletion rate, as used herein can be calculated by sensing the metabolite (O₂) that is consumed from the media sample, which can then be reported in the form of a rate (change in analyte over time) or by measuring, or indirectly assessing, analyte concentration at a preselected timepoint (end point). Conversely, production of oxygen leading to an increase in oxygen concentration can also be determined by measuring the increase in oxygen over time.

Changes in oxygen consumption can be determined in sealed on unsealed systems. In one embodiment, the definition of OCR includes where oxygen consumption is not determined in a sealed system, e.g., a system allows oxygen back diffusion or substantial oxygen back diffusion to the sample, or where oxygen consumption is oxygen depletion in the sample corrected for oxygen back diffusion to the sample, or oxygen consumption is oxygen depletion without being corrected for oxygen back diffusion to the sample, or the oxygen consumption is determined in a sealed system, e.g., a system that does not allow oxygen back diffusion or substantial oxygen back diffusion to the sample, or oxygen consumption equals, or substantially equals, to oxygen depletion in the sample. Furthermore, in one embodiment, oxygen consumption is determined directly or indirectly, e.g., inferred from a measured oxygen gradient, e.g., within a test well, or by measuring oxygen at a preselected timepoint.

Extracellular Acidification Rate (ECAR), as used herein, can be determined using a basal or initial value for proton efflux for the cell sample, e.g., a value based upon a measurement of proton efflux for the cell sample made prior to formation of the reaction mixture and determining a proton efflux rate after formation of the reaction mixture. For instance, (ECAR), as used herein can be calculated by sensing the metabolite (H+) that is either being consumed or produced in the media sample, which can then be reported in the form of a rate (change in analyte over time) or by measuring analyte concentration at a preselected timepoint (end point). In one embodiment, changes in ECAR can be determined in a sealed or unsealed system.

As used herein, the terms “about,” “approximately,” or “generally,” when used to modify a value, indicates that the value can be raised or lowered by 10%, such as, such as 7.5%, 5%, such as 4%, such as 3%, such as 2%, such as 1%, and remain within the disclosed aspect. Moreover, the term “substantially free of” when used to describe the amount of substance in a material is not to be limited to entirely or completely free of and may correspond to a lack of any appreciable or detectable amount of the recited substance in the material. Thus, e.g., a material is “substantially free of” a substance when the amount of the substance in the material is less than the precision of an industry-accepted instrument or test for measuring the amount of the substance in the material. In certain example embodiments, a material may be “substantially free of” a substance when the amount of the substance in the material is less than 10%, less than 9%, less than 8%, less than 7%, less than 6%, less than 5%, less than 4%, less than 3%, less than 2%, less than 1%, less than 0.5%, or less than 0.1% by weight of the material.

### Detailed Description

It is to be understood by one of ordinary skill in the art that the present discussion is a description of exemplary embodiments only and is not intended as limiting the broader aspects of the present disclosure.

Generally speaking, the present disclosure is directed to a microtiter plate assembly that includes a lid having a plurality of projections and a base having a plurality of reciprocating chambers (e.g., wells) corresponding to the respective projections, a force distributing plate, and a magnetic plate adapter. Namely, the present disclosure has unexpectedly found that when an assembly according to the present disclosure having a unique orientation and specific force is used, a SBS and plate reader compatible assembly is provided that minimizes warping across even large microtiter plate assemblies and provides height clearances with extremely low coefficients of variation. Namely, the present disclosure has found that the unique combination of a force distributing plate having a low weight and high stiffness, in combination with a magnetic adapter configured to provide a specific weight of force, allows an assembly to be provided that has greatly reduced warping and a low coefficient of variation in regard to the height clearance of the projections, which will be discussed in greater detail below.

For instance, in one aspect, at least one of the force distributing plate and the magnetic plate adapter are formed at least in part from a material that is naturally magnetic or paramagnetic, such as steel, iron, nickel, cobalt, aluminum, platinum, tungsten, magnesium, combinations thereof, or alloys and derivates thereof, or are coated in a material that provides magnetic properties to the substrate, while also exhibiting sufficient stiffness. Particularly, at least one of the force distributing plate and the magnetic plate adapter are formed from a material that exhibits a low tendency to warp or bend. For instance, in one aspect, at least one of the force distributing plate and the magnetic plate adapter are formed from a material such that at least one outer row exhibits a percent warp of about 30% or less as compared to at least one inner row, such as about 25% or less, such as about 22.5% or less, such as about 20% or less, such as about 17.5% or less, such as about 15% or less, such as about 12.5% or less, such as about 10% or less, or any ranges or values therebetween, such as about 1% to about 30%, such as about 5% to about 25%, such as about 7.5% to about 20%, utilizing a Starret Granite Surface Plate with an electronic digital indicator (2720-0M) to measure point of deflection in outer row(s). However, while it has been so far discussed that only one of the force distributing plate and the magnetic holder may be formed from such a material, it should be understood that, both the force distributing plate and the magnetic plate adapter are both formed from a material that is magnetic and has a high degree of stiffness.

Alternatively, at least one of the force distributing plate or the magnetic holder are formed from a material having a low tendency to warp or bend, but that is not magnetic. In such an aspect, the force distributing plate includes one or more magnets in a location corresponding to the magnets in the magnetic holder. For instance, in one aspect, a paramagnetic material, may provide a low tendency to warp or bend, but does not provide an adequate weight of force. Thus, in one aspect, at least one of the force distributing plate or the magnetic holder is formed from a non-magnetic or paramagnetic material, such as aluminum, and includes one or more magnets, having the sizes and force described below, that correspond to the magnets on the magnetic holder. Further benefits of utilizing non-magnetic or paramagnetic materials with one or more magnets can also be provided, such as heat treating the non-magnetic or paramagnetic material to increase flatness of the force distributing plate prior to assembly. Thus, as used herein, “magnetic properties” may refer to properties exhibited by a magnetic material or a paramagnetic material, or non-magnetic material or paramagnetic material containing one or more magnets

Nonetheless, in addition to having a high degree of stiffness, the magnetic plate adapter, force distributing plate, or combination thereof, also includes at least one, such as at least two, such as at least three, such as at least four, such as eight or less, such as seven or less, such as six or less magnets attached to the magnetic plate adapter. Stated differently, the magnetic plate adapter, force distributing plate, or combination thereof contains a volume of magnets (or magnetic properties) sufficient to provide about 3 kilograms*force (kgf) or more, such as about 3.5 kgf or more, such as about 4 kgf or more, such as about 4.5 kgf or more, such as about 5 kgf or more, such as about 6 kgf or more, such as about 7 kgf or more, such as about 8 kgf or more, such as about 9 kgf or more, such as about 10 kgf or more, such as about 20 kgf or less, such as about 19 kgf or less, such as about 18 kgf or less, such as about 17 kgf or less, such as about 16 kgf or less, such as about 15 kgf or less, such as about 14 kgf or less, or any ranges therebetween, to the assembly. Particularly, the present disclosure has found that when one or more magnets are used according to the above force ranges, the assembly is able to provide sufficient force to prevent warping and a low enough magnetic field to minimize or eliminate interference of a measurement by one or more plate readers.

In addition, as noted above, the present disclosure has found that such a force distributing plate and the magnetic plate adapter may be sized such that they do not increase the lateral or longitudinal size of the microtiter plate adapter, to be compatible with SBS compliance and existing plate readers. However, the force distributing plate and the magnetic plate adapter are also sized to have a lateral and longitudinal size that closely mirror the microtiter plate holder in order to provide stabilization to the corners and center of the assembly.

For instance, in one aspect, at least one of the force distributing plate and the magnetic plate adapter have a longitudinal length that is about 99.9% or less of a longitudinal length of the lid and/or base, such as about 98% or less, such as about 97% or less, such as about 96% or less, such as about 95% or less, such as about 90% or more, such as about 91% or more, such as about 92% or more, such as about 93% or more, such as about 94% or more, or any ranges or values therebetween. However, it should be understood that, in one aspect, at least one of the force distributing plate and the magnetic plate adapter have a longitudinal length that is generally equal to the longitudinal length of the lid and/or base.

Similarly, in one aspect, at least one of the force distributing plate and the magnetic plate adapter have a lateral width that is about 99.9% or less of a lateral width of the lid and/or base, such as about 98% or less, such as about 97% or less, such as about 96% or less, such as about 95% or less, such as about 90% or more, such as about 91% or more, such as about 92% or more, such as about 93% or more, such as about 94% or more, or any ranges or values therebetween. However, it should be understood that, in one aspect, at least one of the force distributing plate and the magnetic plate adapter have a lateral width that is generally equal to the lateral width of the lid and/or base. For instance, in one aspect, at least one of the force distributing plate and the magnetic plate adapter have a lateral width from about 90% to about 99.9% of the lateral width of the lid and/or base, such as about 92% to about 98%, such as about 94% to about 97%, or any ranges or values therebetween.

Particularly, the present disclosure has found that by utilizing vertically magnetized magnets affixed to the magnetic plate adapter in a generally perpendicular manner, the magnets may be contained within a skirt of the base and still provide adequate magnetic force through the base and the lid to the force distribution plate without expanding the footprint of the assembly, which will be discussed in greater detail below. Thus, the assembly of the present disclosure does not require the lateral width or longitudinal length of the assembly to be increased beyond a standard-sized microtiter plate assembly. For example, when using a ninety-six (96) well microtiter plate assembly, the total footprint of the assembly according to the present disclosure may have a length of about 128 mm or less, a width of about 86 mm or less, and a height of about 20 mm or less, such as a length of from about 125 mm to about 135 mm, such as from about 127 mm to about 130 mm, a width of from about 80 mm to about 90 mm, such as about 84 mm to about 88 mm, and a height from about 15 mm to about 25 mm, such as from about 19 mm to about 23 mm, or any ranges or valued therebetween.

Furthermore, in one aspect, in order to allow the assembly to be compatible with existing plate readers and SBS compliance, at least one of the force distributing plate and the magnetic plate adapter has a height of about 10 mm or less, such as about 9 mm or less, such as about 7 mm or less, such as about 5 mm or less, such as about 4 mm or less, such as about 3.5 mm or less, such as about 3 mm or less, such as about 2.5 mm or less, such as about 2 mm or less, or any ranges or values therebetween. For instance, in one aspect, at least one of the force distributing plate and the magnetic plate adapter have a height of about 1 mm to about 10 mm, such as about 1.5 mm to about 7.5 mm, such as about 1.75 mm to about 4.25 mm, or any ranges or values therebetween.

In one aspect, both of the force distributing plate and the magnetic plate adapter have heights according to the above ranges, or alternatively, both of the force distributing plate and the magnetic plate adapter have a total height of about 12 mm or less, such as about 11 mm or less, such as about 10 mm or less, such as about 8 mm or less, such as about 6 mm or less, such as about 5.5 mm or less, such as about 5 mm or less, such as about 4.5 mm or less, such as about 4 mm or less, or any ranges or values therebetween. For instance, in one aspect, the force distributing plate and the magnetic plate adapter have a total height of about 3.5 mm to about 12 mm, such as about 3.75 mm to about 11 mm, such as about 3.75 mm to about 4.25 mm, or any ranges or values therebetween.

In addition, the present disclosure has found that the sufficient force can be provided solely by a clamping-type function provided by the magnetic force discussed above. Thus, in one aspect, at least one of the force distributing plate and the magnetic plate adapter have a weight of about 600 g or less, such as about 550 g or less, such as about 500 g or less, such as about 450 g or less, such as about 400 g or less, such as about 350 g or less, such as about 300 g or less, such as about 250 g or less, such as about 200 g or less, or any ranges or values therebetween. For instance, in one aspect, at least one of the force distributing plate and the magnetic plate adapter have a weight of about 150 g to about 650 g, such as about 175 g to about 625 g, such as about 190 g to about 610 g, or any ranges or values therebetween.

Furthermore, in one aspect, both the force distributing plate and the magnetic plate adapter have a combined weight of about 800 g or less, such as about 750 g or less, such as about 700 g or less, such as about 650 g or less, such as about 600 g or less, such as about 550 g or less, such as about 500 g or less, such as about 450 g or less, such as about 400 g or less, or any ranges or values therebetween. For instance, in one aspect the force distributing plate and the magnetic plate adapter have a total combined weight of about 350 g to about 850 g, such as about 375 g to about 750 g, such as about 390 g to about 500 g or less, or any ranges or values therebetween.

In one aspect, the force distributing plate and/or the magnetic plate adapter may be formed from a material that naturally has a weight according to the above due to its size and/or thickness. However, in one aspect, the present disclosure has found that by using a material having a high degree of stiffness, a plurality of apertures can be formed in at least one of the force distributing plate and the magnetic plate adapter without compromising on the force and stiffness characteristics described above. Thus, in one aspect, at least one of the force distributing plate and the magnetic plate adapter have a plurality of apertures penetrating through the thickness of the respective force distributing plate and magnetic plate adapter. Particularly, in one aspect, the plurality of apertures may be spaced and/or sized to view each well from a top-down or bottom-up view. In such an aspect, each aperture may correspond one-to-one with each well, or may provide a window through which a plurality of wells are viewed. For instance, in one aspect, a window aperture may provide a view of about 1 or more wells, such as about 2 or more wells, such as about 4 or more wells. Additionally, in an aspect, the force distributing plate may have apertures that are shaped and sized to provide a view of each well individually, whereas the magnetic plate adapter is provided with window apertures. Nonetheless, it should be clear to one having skill in the art that apertures may be selected to provide the selected weight, stiffness, and viewing to the sample in order to allow the assembly to be used with existing plate readers.

Furthermore, as discussed above, the present disclosure has found that by utilizing an assembly according to the present disclosure, the projections or stoppers described herein exhibit a highly accurate height clearance (the distance between the most distal point of the distal end the lower surface of the chamber or well)\, which allows improved sensitivity to measurements of any measurable analyte that are dependent upon sample volume, such as oxygen consumption rate or extracellular acidification for example only, without disrupting cell growth, and in one aspect, the improved consistency is exhibited from well-to-well, row-to-row, column-to-column, or a combination thereof. Particularly, a microtiter assembly according to the present disclosure may have an average height clearance between the most distal point of each projection and the lower surface of the chamber or well when a stop tab of a lid is contacting the stop stab receiver of a base containing the plurality of chambers, of about 0.25 mm or less, such as about 0.20 mm or less, such as about 0.15 mm or less, such as about 0.1 mm or less, such as about 0.075 mm or less, such as about 0.5 mm or less, or any ranges or values therebetween. For instance, in one aspect, the microtiter assembly has an average height clearance of about 0.025 mm to about 0.25 mm, such as about 0.035 mm to about 0.15 mm, such as about 0.045 mm to about 0.1 mm, or any ranges or values therebetween.

Furthermore, as the assembly including the lid and above described projections allow for highly consistent height clearance, in one aspect, the height clearance may have a very low degree of variability, such that the variation in height clearance between adjacent projections/wells is about 10% or less, such as about 5% or less, such as about 2.5% or less, such as about 2% or less, such as about 1% or less. Particularly, the present disclosure has found that when a microtiter assembly is formed according to the above, increased well-to-well, row-to-row, column-to-column, or a combination thereof, sensitivity is achieved even with reduced sample sizes, as the force distributing plate and the magnetic plate adapter provide even force along the entirety of the assembly, allowing each stop tab to properly contact the base and dispose the projections fully into each respective well.

Nonetheless, as discussed above, the assembly according to the present disclosure also provides the assembly with decreased warping, which are discussed in greater detail in the examples below. Particularly, the assembly according to the present disclosure may exhibit warping in an amount of about 25% or less, such as about 20% or less, such as about 15% or less, such as about 10% or less, as discussed above in regard to percent warp, where, as will be discussed in greater detail, flatness or warp measurements are made during stress testing, where the part was cycled between room temperature and 37° C. In addition, the above warping values may be exhibited when tested for about 30 minutes or more, such as about 45 minutes or more, such as about 60 minutes or more, such as about 75 minutes or more, such as about 90 minutes or more, such as about 105 minutes or more, such as up to about 120 minutes.

As may be apparent to one having skill in the art, the assembly of the present disclosure can provide a benefit to any microtiter well and lid combination where a consistent sample size (e.g. consistent distance between sensor and sample or bottom of well), as well as any measurable analyte. For instance, exemplary microtiter equipment that may benefit by use in an assembly according to the present disclosure can generally be shown by U.S. Patent App. Pub. No. 2018/0292393, U.S. Patent App. Pub. No. 2020/0166529, and U.S. Patent App. Pub. No. 2014/0248650, which are incorporated herein in their entirety. Of course, as discussed, it should be acknowledged that other microtiter equipment may also benefit from use in an assembly according to the present disclosure.

Furthermore, in one aspect, a microtiter well and lid that could benefit from use in an assembly of the present disclosure can include a lid having a plurality of projections extending longitudinally from a lid, each projection having a combination of a radial notch and a canted distal end, that can be formed from, or coated with, a material having a low oxygen transmission rate, allowing ambient oxygen to be reduced while also reducing sample size.

Particularly, in one aspect, the present disclosure has found that by utilizing a specifically sized radial notch that extends in a lengthwise manner from the distal end to the proximal end of each projecting portion, air bubbles present in the sample chamber (e.g., a well) are evacuated while also preventing overspilling of the sample. In one aspect, the radial notch, which will be discussed in greater detail in regards to the figures below, has a radial cross-section of about 0.575 mm² or greater, such as about 0.585 mm² or greater, such as about 0.595 mm² or greater, such as about 0.6 mm² or greater, such as about 0.65 mm² or greater, such as about 0.7 mm² or greater, such as about 0.75 mm² or greater, such as about 0.85 mm² or greater, such as about 0.95 mm² or greater, such as about 1.00 mm² or greater, such as about 1.1 mm² or greater, such as about 1.2 mm² or greater, such as about 1.3 mm² or greater, such as about 1.4 mm² or greater, such as about 1.5 mm² or greater, such as about 1.6 mm² or greater, such as about 1.7 mm² or greater, such as about 1.75 mm² or greater, such as about 1.8 mm² or greater, such as about 1.9 mm² or greater, such as up to about 2 mm² or less. For instance, in one aspect, the radial notch has a radial cross-section from about 0.85 mm² to about 2.5 mm², such as from about 1 mm² to about 2.25 mm², or from about 1.25 mm² to about 2 mm², or any ranges or values therebetween As discussed above, the present disclosure has surprisingly found that when the radial notch has a radial cross section sized according to the above, the radial notch is able to allow escape of air bubbles from the sample without contributing to re-oxygenation.

Stated differently, in one aspect, the radial notch may have a cross-sectional area that is proportional to the cross-sectional area of the proximal end of the projection. For instance, the proximal end of the projection can define a cross-sectional area including the cross-sectional area defined by the radial notch. Thus, the radial cross-section of the radial notch may account for about 5% or more of the total cross-sectional area of the proximal end of each projection, such as about 8% or more, such as about 10% or more, such as about 15% or more, such as about 20% or more, such as about 25% or more, up to about 30% or less, such as about 5% to about 30%, such as about 8% to about 25%, or such as about 10% to about 20%, or any ranges or values therebetween. As discussed above, the present disclosure has surprisingly found that when the radial notch has a radial cross section sized according to the above, the radial notch is able to allow escape of air bubbles from the sample without contributing to re-oxygenation or overspilling. While any diameter projection may be used in accordance proportional to a notch as discussed above, in one aspect, at least one projection has a diameter of about 1 mm to about 5 mm, such as about 1.5 mm to about 4 mm, such as about 2 mm to about 3 mm, or any ranges or values therebetween.

Furthermore, the radial notch may have any cross-sectional shape, such as circular, elliptical, square, triangular, nodular or the like. However, in one aspect, the radial notch may have a circular cross section, such as circular or oval. Thus, in such an aspect, and as will be discussed in greater detail in regard to the figures below, the radial notch extends into the sidewall of each projection along the entire length of the respective projection, from distal end to proximal end, without penetrating or piercing the sidewall of the projection. Stated differently, the radial notch forms a portion of the sidewall of each projection and does not extend into an interior portion of the projection. Therefore, it should be clear that any notch or channel formed by the radial notch is formed between the portion of the sidewall formed by the radial notch and a sidewall of a chamber or well conforming to the respective projection, and is not formed in an interior (e.g., hollow) of the projection.

In addition, by using a radial notch having the above sizing and dimensions, the radial notch allows excess sample to be contained between the respective projection body and a side wall of the sample chamber or well without overspilling the chamber or well and contaminating adjacent chamber(s) or well(s). Thus, in such an aspect, the distal end of the projection is at least partially contacted by the sample or is fully immersed in the sample such that some sample is contained in a channel formed between the radial notched and the respective conforming chamber or well. However, it should be noted that in one aspect, each projection has only one notch (whether for dissipation of oxygen or overspilling for other use). Namely, when shaped and sized according to the above, only a single radial notch is needed to dissipate bubbles and provide an overspill containment area. However, in a further aspect, one or more of the projections can contain two notches, three notches, or four notches. In such an aspect, each notch may be shaped and sized as discussed herein. As will be discussed in greater detail below, in one aspect, at least one pillar has two notches formed symmetrically across a center line extending from the proximal end to the distal end of the pillar.

In one such aspect where the projection includes at least two notches, the distal end of the projection can contain a spotting surface that is generally parallel to a lower surface of the respective well or the plane of the lid. For instance, the spotting surface may have generally the same cross-sectional shape as the respective projection, and have a diameter of about 0.5 mm to about 4.5 mm, such as about 1 mm to about 4 mm, such as about 1.5 mm to about 3.5 mm, or any values or ranges therebetween. Furthermore, in one aspect where one or more of the lid, projection, base, or wells are formed to have a non-clear color, the spotting surface may be generally transparent or frosted.

However, while the sample can fully encircle or contact the distal end of at least some, or all, of the projections in one aspect, it should also be understood based upon the description of the method for determining oxygen consumption rate that the oxygen consumption rate is not determined based upon a visual inspection from above the sample. Instead, the oxygen consumption rate is determined utilizing a signal detector, such as a plate reader, in one aspect, which measures the photoluminescent material, which will be discussed in greater detail below. Therefore, in one aspect, the substrate material used to form some or all of the plurality of projections, cover lid, or base containing chambers or wells corresponding to the plurality of projections is formed from a translucent material, which is differentiated from a transparent material, by having limited viewing of the sample through the material while still allowing light to pass sufficient to enable probe interrogation. Particularly, such a material can diffuse light while allowing the photoluminescent material to be measured according to the above method from a top-down plate reader, bottom-up plate reader, or a combination thereof, as known in the art. In one aspect, at least one of the base, the lid, and one or more projections can be translucent and have a black, white, or frosted appearance, or can be transparent.

Furthermore, the distal end of each longitudinal projection (e.g., the end of each projection opposite the end adjacent to the lid) can be canted or sloped at a specific angle relative to a plane parallel to the lid (or well bottom) such that the highest side of the distal end (e.g. the side of the distal end that has a shorter length from the distal end to the lid) is adjacent to the radial notch, and any air bubbles present in the sample are guided to the notch, facilitating removal of the air bubbles. Therefore, in one aspect, the distal end is canted or sloped at an angle of about 5° or greater, such as about 7.5°, such as about 10° or greater, such as about 12.5° or greater, such as about 15° or greater, such as about 20° or greater, such as about 25° or greater, up to about 30° or less, relative to the lid or well bottom. For instance, in one aspect, the distal end is canted or sloped at an angle of about 3° to about 30°, such as about 3° to about 25°, such as about 5° to about 20°, or any ranges or values therebetween. Particularly, when angles according to the above are used for the distal end canted towards the radial notch, bubbles may be effectively removed from the sample while also allowing a highly sensitive oxygen consumption rate reading at small samples sizes.

Moreover, as may be understood, in an aspect contain one or more projections having two or more notches, the projection(s) may also contain two or more angled portions at the distal tip corresponding to the two or more projections. For example, in an aspect containing two projections, the canted distal tip can include two angled portions each having a highest side adjacent to a respective radial notch and having and each having a most distal point that meet at approximately the longitudinal center line. In such a manner, bubbles may be effectively removed to one or more notches for removal from the sample.

For instance, a lid according to the present disclosure can be used to measure oxygen consumption rate, among other measurements, utilizing fill volumes of 100 μL or less, such as about 90 μL or less, such as about 80 μL or less, such as about 70 μL or less, such as about 60 μL or less, such as about 60 μL or less, such as about 50 μL or less, such as about 40 μL or less, such as about 30 μL or less, such as about 25 μL or less, such as about 20 μL or less, such as about 15 μL or less, such as about 10 μL or greater, such as about 15 μL or greater, or any ranges or values therebetween. For instance, in one aspect, the fill volume may be about 10 μL to about 100 μL, such as about 15 μL to about 90 μL, such as about 20 μL to about 80 μL, or any ranges or values therebetween.

Stated another way, plate lids according to the present disclosure allow lower seeding densities to be used while maintaining measurable results. For instance, in one aspect, the plate lid according to the present disclosure can be utilized with seeding densities of about 100,000 cells or less, such as about 75,000 cells or less, such as about 50,000 cells or less, such as about 25,000 cells or less, such as about 12,500 cells or less,, such as about 10,000 cells or less, such as about 7,500 cells or less, such as about 5,000 cells or less, such as about 2,500 cells or less such as about 1,000 cells or greater, such as about 2,500 cells or greater, or any values or ranges therebetween. For instance, in one aspect, the seeding density is from about 2,500 cells to about 125,000 cells, such as about 6,500 cells to about 90,000 cells, such as about 10,000 cells to about 75,000 cells, or any ranges or values therebetween.

Namely, as mentioned above, by utilizing an assembly according to the present disclosure, increases sensitivity and improved measurement consistency can be achieved. Thus, the assembly according to the present disclosure can provide benefits to fluid samples containing a variety of cell lines, for instance, due to increased sensitivity and reduced oxygen ingress/back diffusion. Namely, as discussed above, the assembly according to the present disclosure can be used with adherent cell lines as known in the art, in addition to suspension cell lines. As understood in the art, suspension cell lines can be immobilized on a coated plate (e.g., by use of a centrifuge and an immobilization coating such as PDL, PLL, collagen, and fibronectin for example, which will be discussed in greater detail below) and be assayed using any one or more of the methods described herein.

Furthermore, the assembly of the present disclosure can also be utilized with and provide benefits to 3D constructs. Examples of such constructs include spheroids (examples of which may be described in U.S. Provisional Patent Application No. 63/276,099, which is incorporated herein in its entirety) and/or organoids immobilized on coated plates, 3D culture systems utilizing scaffold or matrix materials (e.g. electrospun fibrous scaffolds, collagen, hydrogel, and Matrigel, including Lonza's RAFT™ 3D cell culture system), tissues and microtissues, and magnetic spheroids.

For instance, one such magnetic spheroid method that can benefit from small sample sizes includes a magnetic spheroid drive. As known in the art, magnetic spheroids can be measured using various methods in combination with low adhesion plates, magnetic particles, and magnets, for example, or may be localized (e.g. using poly lysine, a magnetic array, or the like). An example of one spheroid workflow, which is for example only, includes the Greiner workflow, where cells are magnetized, and formed into a spheroid utilizing a magnetic drive. After spheroid formation, the spheroids can be kept in the Greiner spheroid plate or moved to another suitable microplate. Regardless of the plate selected, a magnetic drive can be used to localize the spheroid while inserting the lid according to the present disclosure in order to orient the spheroid at the base of the well. Furthermore, in one aspect, the magnetic drive may be maintained during the assay to localize the spheroid.

Additionally or alternatively, as noted above, one or more of the projections may be hollow. Thus, in one aspect, the hollow core of one or more projections can contain a core magnet to aid in spheroid localization, allowing the lid to be placed without a magnetic drive. In one aspect, one or more of the projections contain a core magnet that is permanently affixed to the respective hollow. Conversely, in one aspect, the lid assembly according to the present disclosure may include a projection overlay that is disposed between the projections and the cover block. The projection overlay includes one or more magnetic spines that extend into the hollow of one or more projections in a releasable manner, and can be removed after spheroid localization or spheroid transport. In one such aspect, the projection overlay can include a number of magnetic spines corresponding to the number of projections, such that when the projection overlay is placed, each projection hollow is at least partially occupied with a magnetic spine.

Moreover, as may be understood by one having skill in the art, it should be understood that other fluid samples may benefit from a lid according to the present disclosure. For instance, the present disclosure can be used in conjunction with enzymes, isolated organelles, such as mitochondria, microscopic organisms, prokaryotes, eukaryotes, stem cells, cardiomyocytes, ex vivo samples embedded on microscope slides and the like. Nonetheless, while examples of various cell lines and samples have been provided, it should be understood that, in one aspect, any sample suitable for interrogation using a chamber or well according to the present disclosure that benefits from tailored sensitivity, reduced or controlled oxygen ingress/back diffusion, reduced sample volume (such as increased cell to volume ratio), or combinations thereof, may be used according to the present disclosure.

Furthermore, as may be understood by one having skill in the art, one or more culture coatings may be used on the plate, projection, lid, or combination thereof, based upon the fluid sample selected. For instance, as discussed above coatings for suspension cell lines may include a poly-D-lysine (PDL), poly-l-lysine (PLL) collagen, and fibronectin. Moreover, for spheroid assays, an ultra-low attachment coating, other common tissue culture coatings, or combinations thereof may be used. For instance, suitable ultra-low attachment coatings include BioFloat® Flex available from faCellitate and Lipidure® available from NOF Corporation which aid in avoiding unspecific surface binding of proteins and cells. However, it should be clear that other culture coatings may be used as known in the art.

Nonetheless, in one aspect, the lid according to the present disclosure is in reversible contact with the base and is therefore may not be permanently affixed to the base. Namely, each projection can be in reversible association with the respective chamber or well. For instance, in one aspect, the stop tab of the lid can maintain contact with the base during the desired testing period using the plate assembly described herein.

To achieve the reversible attachment, the present disclosure has found that a specific wall clearance, defined as the distance between any portion of the side walls of each projection to the side wall of the chamber or well, is used to enable the lid to be removed while returning excess sample to the chamber or well. Namely, if too small of a wall clearance is used, the lid will not be removable, but if the wall clearance is too large, air bubbles become trapped between portions of the side wall that do not contain the radial notch, reducing the ability for air bubbles to be fully evacuated. Therefore, in one aspect, the assembly according to the present disclosure has a wall clearance of about 0.5 mm or less, such as about 0.4 mm or less, such as about 0.3 mm or less, such as about 0.2 mm or less, such as about 0.175 mm or less, such as about 0.15 mm or less, or any ranges or values therebetween. For instance, in one aspect, the wall clearance is from 0.025 mm to about 0.5 mm, such as about 0.05 mm to about 0.4 mm, such as about 0.75 mm to about 0.3 mm, or any ranges or values therebetween.

Stated differently, the wall clearance should be significantly smaller than the clearance by the radial notch in order to evacuate air bubbles through the notch instead of trapping air bubbles between the side wall of the projection and side wall of the corresponding well. Therefore, in one aspect, the radial notch defines a radial notch clearance defined as a distance between the point along the radial cross section that has the largest distance from the sidewall of the respective well (e.g. the apex of the radial notch) and the respective well sidewall, wherein the wall clearance is about 50% of the radial notch clearance or less, such as about 40% or less, such as about 30% or less, such as about 20% or less, such as about 10% or less, such as about 5% or less, such as about 2.5% or less, such as about 1% or less of the radial notch clearance.

Furthermore, as the lid assembly including the lid and above described projections allow for highly consistent wall clearance, in one aspect, the wall clearance may have a very low degree of variability, such that the variation in wall clearance between adjacent projections/wells is about 10% or less, such as about 5% or less, such as about 2.5% or less, such as about 2% or less, such as about 1% or less. Particularly, the present disclosure has found that when a microtiter assembly is formed according to the above, increased sensitivity is achieved even with reduced sample sizes. However, it should be understood that, in one aspect, the lid may be permanently affixed to the base once placed in association with the base, such as an aspect where the assembly is not intended for reuse.

Regardless of the method used, a further advantage of the assembly of the present disclosure is that evaporation is limited due to the due to the small wall clearance, while remaining reversible. Thus, unlike fixed or permanent lids, as well as samples utilizing oil-in-headspace, samples according to the present disclosure may undergo further testing after the cellular respiration measurements are complete. For instance, the assembly of the present disclosure would enable protein assays or fluorescent based assays to be completed after oxygen consumption rate testing, such as a post-assay immunofluorescence assays (including TOM 20 and LC-3, for example).

Nonetheless, it should also be understood that in addition to subsequent testing, additional simultaneous testing may be conducted in conjunction with the fluorescence based assay, which may also be referred to as “multiplexing”. Namely, as will be discussed in greater detail, as the assembly of the present disclosure is Society for Biomolecular Screening (SBS) compatible, and is therefore compatible with existing signal detectors for measuring additional target analytes (in addition to H+, CO, CO₂, O₂ discussed in greater detail herein) including calcium, ATP, NADP/NADHP, temperature, mitochondrial membrane potential, reactive oxygen species, or the like. Thus, while it should be understood by one having skill in the art that any additional testing may be conducted simultaneously that does not interfere with the target analyte may be multiplexed, in one aspect, additional testing can include stains (such as Hoechst and Calcein AM), florescent dyes, mitochondrial membrane potential (JC-1, e.g. TMRE/TMRM), reactive oxygen species (DHE), or combinations thereof.

Alternatively, the multiplexing analyte may require the use of a separate signal detector that does not interfere with the target analyte(s) discussed herein. The additional signal detector can have one or more detection modes, such as detection of absorbance, fluorescence intensity, luminescence, time-resolved fluorescence, fluorescence polarization, imaging, including florescence imaging, or combinations thereof. Plate readers having those detection modes are commercially available. Alternatively, in an aspect, the sensors can be interrogated from below the sensors, such as from below the lid of the apparatus; in such arrangements, the signal should be able to pass through the well and/or chamber so that it can be detected, for example, by having the well and/or chamber include a transparent material or translucent material, or being able to be interrogated from above without requiring a transparent material on a distal end of the projection, such as having one or more fiber optics passing through or contained within the lid and/or projections.

In addition, as briefly mentioned above, and as may be understood by one having skill in the art, the substrate material forming the base, lid, projections, or a combination thereof has increased contact with the sample due to the ability to overfill the chamber as well as the lack of oil barrier between the sample and the headspace or lid. Thus, as may be apparent to one having skill in the art, the assembly of the present disclosure allows the sample volume and/or fill volume, substrate material, and optional low oxygen transmission coating to be selected based upon the cell line to be studied. Namely, it is well known that different cells respire differentially, and may therefore deoxygenate the sample at different rates. For instance, in one aspect, cells that respire quickly, may require a larger fill volume or may have less sensitivity to the substate material or coating, whereas a cell line that is less active may benefit from a smaller sample volume and a coating or substrate material that limits back diffusion of oxygen. Nonetheless, in one aspect, the material used to form the substrate or to coat the substrate has an oxygen transmission rate of about 600 cm³/m²/24 hours or less, such as about 300 cm³/m²/24 hours or less, such as about 150 cm³/m²/24 hours or less, such as about 120 cm³/m²/24 hours or less, such as about 100 cm³/m²/24 hours or less, such as about 80 cm³/m²/24 hours or less, such as about 50 cm³/m²/24 hours or less, such as about 25, cm³/m²/24 hours or less, such as about 16 cm³/m²/24 hours or less, down to about 0 cm³/m²/24 hours or less or more measured at 23° C. and 0% RH according to ASTM D 3985.

In addition, in one aspect, as will be discussed in greater detail in the examples below, when low oxygen back diffusion is needed for sensitive samples, the material used to form the substrate or to coat the substrate has a low oxygen transmission rate of about 16 cm³/m²/24 hours or less, measured at 23° C. and 0% RH according to ASTM D 3985, such as about 15 cm³/m²/24 hours or less, such as about 12.5 cm³/m²/24 hours or less, such as about 10 cm³/m²/24 hours or less, such as about 7.5 cm³/m²/24 hours or less, such as about 5 cm³/m²/24 hours or less, such as about 2.5 cm³/m²/24 hours or less, such as about 1 cm³/m²/24 hours or less, such as about 0.5 cm³/m²/24 hours or less, or any ranges or values therebetween. For instance, in one aspect, the material has an oxygen transmission rate from about 0.01 cm³/m²/24 hours to about 20 cm³/m²/24 hours, such as about 0.05 cm³/m²/24 hours to about 17.5 cm³/m²/24 hours, such as about 0.1 cm³/m²/24 hours to about 15 cm³/m²/24 hours, or any ranges or values therebetween. Such low-oxygen back diffusion may aid in applications such as cancer research, as the necessary cell lines require low cell densities which are enabled by use of a lid according to the present disclosure as discussed herein.

For instance, in one aspect, any portion of the assembly may be formed from a substrate material having a variety of forms and compositions and may derive from naturally occurring materials, naturally occurring materials that have been synthetically modified, or synthetic materials. Examples of suitable substrate materials include, but are not limited to, nitrocellulose, glasses, silicas, teflons, metals (for example, gold, platinum, and the like), and ceramics (including aluminum oxide, silicon oxide, and the like), composites, and laminates thereof. Suitable substrate materials also include polymeric materials, including polysaccharides such as agarose (e.g., that available commercially as Sepharose®, from Pharmacia) and dextran (e.g., those available commercially under the tradenames Sephadex® and Sephacyl®, also from Pharmacia), polyacrylamides, polystyrenes, polyvinyl alcohols, copolymers of hydroxyethyl methacrylate and methyl methacrylate, polyesters, including poly(ethylene terephthalate) and poly(butylene terephthalate); polyamides, (such as nylons); polyethers, including polyformaldehyde and poly(phenylene sulfide); polyimides, such as that manufactured under the trademarks KAPTON (DuPont, Wilmington, Del.) and UPILEX (Ube Industries, Ltd., Japan); polyolefin compounds, including cyclis olefine polymer, ABS polymers, Kel-F copolymers, poly(methyl methacrylate), poly(styrene-butadiene) copolymers, poly(tetrafluoroethylene), poly(ethylenevinyl acetate) copolymers, poly(N-vinylcarbazole), polytetrafluoroethylene, polypropylene, polystyrene, polycarbonate, cyclo-olefin polymers (COP), such as sold under the brand name Zeonor® and Zeonex®, as well as cyclo-olefin copolymers (COC), such as sold under the brand name Topas®, and blends thereof, and the like. Certain polymeric materials that may be used for substrate materials include organic polymers that are either homopolymers or copolymers, naturally occurring or synthetic, crosslinked or uncrosslinked.

For instance, in one aspect, the substrate material for all or a portion of the lid and base may be selected to be a material having a naturally low oxygen transmission rate, such as an acrylate polymer, which can be polymethylmethacrylate in one aspect, polyethylene terephthalate, alone or further coated with a polymer having a very low oxygen transmission rate. In one aspect, any of the above substrate material can be used, but at least a portion of the substrate material is coated with a polymeric or ceramic compound, such as a glass compound, or any compound suitable with low-temperature deposition processes, such as Al₂O₃ or SiO₂, atomic layer deposition (ALD), molecular vapor deposition, or plasma-enhanced chemical vapor deposition (PECVD), such as ceramics and polymers, including silicon oxide, silicon nitride, silicon oxynitride, aluminum oxide, mixed oxides, flexible glass (Al₂O₃ layers alternated with polyl-acrylate layers on a PEN-based substrate, available as ClearProtect© 3D barrier coating from Antec) having a low oxygen transmission rate. Other surface treatments may be generally available include Actiplas, Aquacer, Carbocer, Lipocer, Plasmaclean, Silitec, or combinations thereof. Furthermore, in one aspect, all of a sample contacting portion of the plurality of projections are coated in a material having a very low oxygen transmission rate, alone or in combination with coating other portions of the assembly. Particularly, such a coating may further aid in preventing re-oxygenation of the sample after elimination of the gaseous headspace.

Furthermore, it should be understood by one having skill in the art that additional reagents may be stored on the lid and/or projection, either by lyophilizing or solubilizing the reagent in a reagent substrate in order to further tune the assay. Exemplary reagent substrates include D-glucose-6-PO4, Pyruvic Acid, α-Keto-glutaric Acid, α-Keto-Butyric Acid, Ala-Gin, Sparker Malate Control, α-D-Glucose, citric acid, succinic acid, D,L-β-Hydroxy-Butyric Acid, L-Serine, Acetyl-L-Carnitine, γ-Amino-butyric acid, glycogen, D,L-α-Glycerol-PO4, D,L,-Isocitric Acid, Fumaric Acid, L-Glutamic Acid, L-Ornithine, Octanoyl-L-Carnitine, α-Keto-Isocaproic Acid, D-Glucse-1-PO4, L-Lactic Acid, cis-Aconitic Acid, L-Malic Acid, L-Glutamine, Tryptamine, Palmitoyl-D, L-Carnitine, L-leucine, and combinations thereof. Furthermore, exemplary reagents can include metabolic modulators, such as FCCP, Rotenone, Antimycin A, BAM15, and/or oligomycin, substrate catalyst, enzymes, including glucose oxidase, and exemplary inhibitors include Complex I Inhibitor Rotenone, Complex II Inhibitor Malonate, Complex III Inhibitor Antimycin A, Uncoupler FCCP, Ionophore K Valinomycin, Gossypol, Polymyxin B, Complex I Inhibitor Pyridaben, Complex II Inhibitor Carboxin, Complex III Inhibitor Myxothiazol, Uncoupler 2,4-Dinitrophenol, Calcium, CaCl2, Nordihydro-guaiaretic acid, Amitriptyline, Meclizine, Berberine, Alexidine, Phenformin, Diclofenac, Celastrol, Trifuoperazine, Papaverine, or combinations thereof. However, it should be understood that other reagent substrates, reagents, and inhibitors may be used as known in the art.

As discussed, in one aspect, the assembly according to the present disclosure is directed to a microtiter well plate assembly. Therefore, the plate cover and base may have 6, 8, 24, 96, 384, 1536 and so forth corresponding projections/wells as known in the art. However, in one aspect, the assembly of the present disclosure is particularly well suited for a microtiter plate assembly having 96 wells and corresponding projections or more.

Example aspects of the present disclosure are directed to an assembly and process for analyzing one or more biological constituents, including cellular parameters, contained in or associated with a biological material sample, such as a cell culture. The process and system utilize light detection and ranging components in a manner that not only efficiently takes readings, but also can take faster measurements than many conventional systems. However, it should be understood that, in one aspect, the probe and corresponding sensor may be selected to be any device capable of sensing and reporting changes in a target analyte, such as H⁺, CO, CO₂, O₂, or combinations thereof. Moreover, it should be understood, as discussed above, that the assembly according to the present disclosure is suitable for determining extracellular, intracellular, and pericellular analytes.

For instance, oxygen-sensitive photoluminescent probes capable of sensing and reporting the oxygen concentration of an environment in fluid communication with the probe are widely known. See for example, United States Published Patent Applications 2011/0136247, 2009/0029402, 2008/199360, 2008/190172, 2007/0042412, and 2004/0033575; U.S. Pat. Nos. 8,242,162, 8,158,438, 7,862,770, 7,849,729, 7,749,768, 7,679,745, 7,674,626, 7,569,395, 7,534,615, 7,368,153, 7,138,270, 6,989,246, 6,689,438, 6,395,506, 6,379,969, 6,080,574, 5,885,843, 5,863,460, 5,718,842, 5,595,708, 5,567,598, 5,462,879, 5,407,892, 5,114,676, 5,094,959, 5,030,420, 4,965,087, 4,810,655, and 4,476,870; PCT International Published Application WO 2008/146087; and European Published Patent Application EP 1134583, all of which are hereby incorporated by reference. Such optical sensors are available from a number of suppliers, including Presens Precision Sensing, GmbH of Regensburg, Germany, Oxysense of Dallas, Texas, USA, and Agilent Technologies of Cork, Ireland.

Methods and techniques for sensing of oxygen within a test tube or well of a microtiter plate using oxygen-sensitive photoluminescent probes are widely known as exemplified by WO2012/052068, US Pat. Appln. Pub 2013/0280751 and US Pat. Appln Pub. 2014/0147882, all incorporated herein by reference. These methods and techniques are suitable for use in determining oxygen concentration within a test tube or well sealed with an implement in accordance with the present invention.

However, while it has so far been discussed using a phosphorescent based sensor, it should be understood that optical sensors according to the present disclosure may also utilize solid-state, nanoparticulate, microparticulate, and/or magnetic sensors, or the like. For instance, solid state sensors may include one or more spots or films on the lid, base, projections, or combination thereof, where particle base sensors may generally be in solution or in suspension. Alternatively, in one aspect, particle based sensors can be loaded into cells or coated onto a surface. Nonetheless, such sensors can include optical, O₂, pH, temperature, CO₂, or combinations thereof, as known in the art.

Furthermore, in one aspect, the sensor can be an electrochemical, or potentiometric sensors. Additionally or alternatively, electrodes may also be included in the well in order to measure electrical characteristics, including impedance. Notwithstanding the senser selected, in one aspect, and as discussed above, it should be understood that the well or chamber may also contain one or more reference probes which generates a signal of known value for instrument calibration in the form of any of the sensors discussed above.

Regardless of the type of solid sensor selected, in one aspect for example only, the sensor may be embedded in a permeable medium, such as a permeable medium selected from hydrogel, silicone, and Matrigel. In some aspects, the sensor is attached at least one of the projections by solidifying or removing the medium (such as by drying, curing, cooling, evaporating or other technique). The solid-state sensor can be applied by dipping or spotting the distal end of at least one of projections in a mixture of a fluorescent indicator in a medium.

However, it should be appreciated that in certain aspects, the sensor can be spotted or dipped onto all or a portion of one or more of the projections. It should further be appreciated that in certain aspects, the sensor can be removably connectable to the body of one or more projections of the assembly. It should further be appreciated that in certain aspects, the sensors can be integrally formed with one or more projections. Integrally forming the sensors on one or a plurality of projections can be achieved by one or more techniques, such as vapor deposition, chemical coating, spin coating, dipping, and robotic spotting.

The assembly and processes according to example aspects of the present disclosure can be well suited to measuring constituents in all different types of samples, such as biological samples. In one aspect, for instance, the systems and processes according to example aspects of the present disclosure can be used to measure one or more constituents or a parameter related to the constituent in cellular material. The one or more constituents may be contained in a medium surrounding the cells or can be contained within the cells themselves. In some embodiments, the biological sample being tested may contain cellular material derived from cells, such as cellular organelles, mitochondria, or cellular extracts. Of particular advantage, the measurements can be completed in a label-free manner.

In one aspect, the cell sample is obtained or derived from a subject, such as a human or non-human animal. In one aspect, the subject is a mouse, which, in an aspect, has, or is at risk of having, a disorder. Nonetheless, in an aspect, the cell sample can include a primary cell, a cell isolated or harvested directly from a living tissue or organ, a cultured cell, and/or an immortalized cell. For instance, the cell sample can include a primary cell, or a cell isolated or harvested directly from a living tissue or organ, and then cultured ex vivo. In an aspect, the cell sample includes a cell that has been modified, e.g., genetically engineered for heterologous expression of a gene of interest, and/or genetically engineered for inhibition expression of a gene, such as cells from knock out mouse or CRISPR KO libraries. Nonetheless, in one aspect, the cell sample includes a stem cell or a cell derived from a stem cell. Nonetheless, regardless of the cell used, in one aspect, the cell sample includes a medium, e.g., a culture medium or a growth medium, where the cell can be disposed in the medium. Furthermore, as would be understood, in one aspect, the cell sample comprises a plurality of cells, e.g., a plurality of cells described herein.

The cells being tested can comprise any suitable cell sample, including but not limited to cultured cells, primary cells, human cells, neurons, T cells, B cells, epithelial cells, muscle cells, stem cells, induced pluripotent stem cells, immortalized cells, pathogen-infected cells, bacterial cells, fungal cells, plant cells, archaeal cells, mammalian cells, bird cells, insect cells, reptile cells, amphibian cells, and the like. The cells being tested may also comprise three-dimensional cell samples, such as tissue samples, cell spheroids, biopsied samples, cell scaffolds, organs-on-a-chip, and the like. Examples of parameters that may be measured and are related to the above cell functions include carbon dioxide concentration, pH, oxygen concentration or oxygen partial pressure, calcium ions, hydrogen ions, and the like. However, in one aspect, the measured parameter is oxygen concentration, such as oxygen depletion, or related derivative measurement such as oxygen consumption or cellular oxygenation. Through these tests, one can gain an understanding of what drives cell phenotype and function and/or an accurate picture of the cellular environment or microenvironment.

The assembly and process according to example aspects of the present disclosure can be used to measure live cell metabolic data, or (micro) environmental conditions of any viable cell. The cellular material being tested, for instance, can comprise bacteria cells, fungus cells, yeast cells, prokaryotic cells, eukaryotic cells, and the like. Cells that can be tested include mammalian cells including animal cells and human cells. Particular cells that can be tested include cancer cells, immune cells, immortal cells, primary cells, induced pluripotent stem cells, cells infected with viral or bacterial pathogens, and the like.

For example, in one aspect, the assembly and process according to example aspects of the present disclosure can be used to assist in immunotherapy. Immunotherapy is a type of treatment that bolsters a patient's immune system for fighting cancer, infections, and other diseases. Immunotherapy processes, for instance, can include adoptive cell based therapies, such as the production of T cells, natural Natural Killer (NK) cells, monocytes, macrophages, combinations thereof and the like. During T cell therapy, for instance, T cells are removed from a patient's blood. The T cells are then sent to a bioreactor and expanded or cultivated. In addition, the T cells can be changed so that they have specific proteins called receptors. The receptors on the T cells are designed to recognize and target unwanted cells in the body, such as cancer cells. The modified T cells are cultivated in a bioreactor to achieve a certain cell density and then supplied to a patient's body for fighting cancer or other diseases. T cell therapy can also be referred to as adoptive T cell therapy or T-cell transfer therapy, one example of which is referred to chimeric antigen receptor (CAR) T cell therapy. The use of T cells for adoptive T cell therapy or T-cell transfer therapy has recently proliferated due to great success in combating blood diseases. In some embodiments, aspects of the present invention may be used to monitor the health of T cells used in adoptive T cell therapy or T-cell transfer therapy. In some embodiments, aspects of the present invention may be used to monitor T cell activation, T cell exhaustion, T cell metabolism including of starting material and modified products, and the like.

NK cells are a type of cytotoxic lymphocyte that can seek out and destroy infected cells within the body. NK cells can display very fast immune reaction responses. Consequently, the use of NK cells in anticancer therapy has grown tremendously in interest and popularity. There is only a limited number of NK cells in the blood of a mammal, however, requiring that NK cells be grown to relatively high cell densities within bioreactors.

The culturing of cells, such as T cells, NK cells, or other mammalian cells, typically requires a somewhat complex process from inoculation to use in patients. The assembly and process of the present disclosure can be used to monitor cell metabolism during any point in the culturing process to ensure that the cells are healthy, and/or have the desired metabolic phenotype, and that the media in which the cells are growing contains an optimized level of nutrients. The system and process, for instance, can be used to make adjustments for assuring the metabolic fitness of the cells as they are growing.

In addition to immune cells, the metabolism of cancer cells can also be monitored for providing an understanding of which nutrients fuel the cancer cells or how cancer cell metabolism can be targeted or modulated. For example, the assembly and process according to example aspects of the present disclosure can reveal mechanisms or components that impact the metabolism of the cancer cells for inhibiting growth. The system and process of the present disclosure is also well suited for use in toxicology. For instance, the process and assembly of the present disclosure can be used to detect mitochondrial liabilities among potential therapeutics. The risk of mitochondrial toxicity, for instance, can be assessed with high specificity and sensitivity. In this manner, the mechanism of action of some mitochondrial toxicants can be determined.

The systems and processes according to example aspects of the present disclosure can also be used to assist in treating obesity, diabetes, and metabolic disorders by aiding in the discovery of relevant therapies. For instance, the process and system can be used to measure functional effects of genetic changes to metabolic pathway components. Nutrients used in healthy and diseased cell models can be examined. Further, fatty acid oxidation and glycolysis can be assessed in different cell types, including stem cells.

Nonetheless, aspects of the present disclosure may be further understood according to the discussion of the following figures.

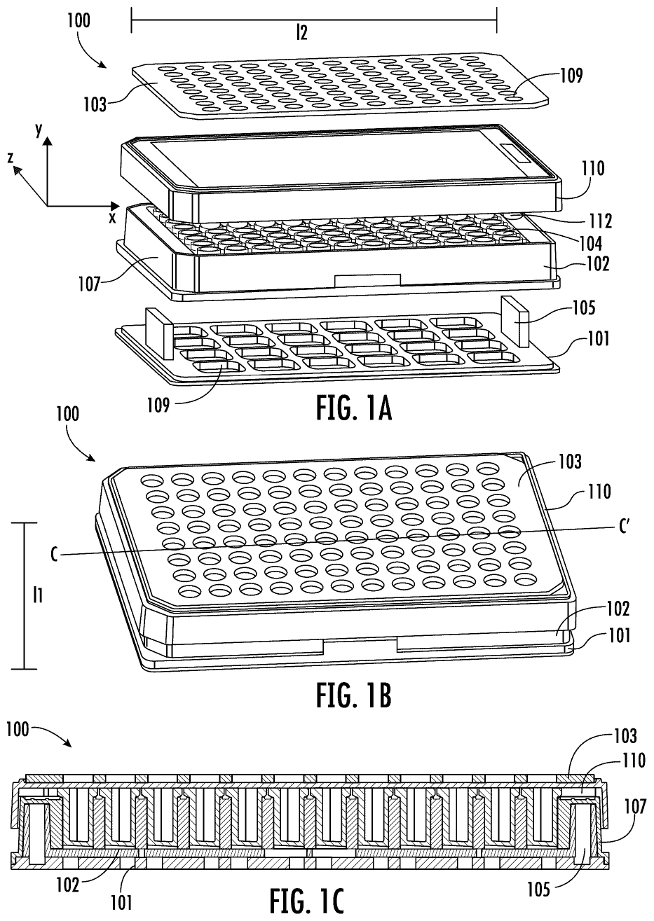

Referring first to FIGS. 1A-1C, a microtiter plate assembly 100 according to the present disclosure is illustrated. Namely, the assembly 100 includes a magnetic plate adapter 101, a base 102, a lid 110, and a force distributing plate 103. As shown in FIGS. 1A-1C, the magnetic plate adapter 101 has a high degree of flatness in the x or horizontal direction, with magnets 105 attached in a perpendicular direction (e.g., y-direction or vertically extending from the magnetic plate adapter 101). By orienting the magnets 105 in such a manner, they may be seated within the apron 107 of the base 102, such that the length of the base in the z-direction l1 (shown more clearly in FIG. 1B) or the longitudinal or x-direction l2 of the magnetic plate adapter 101 does not extend past that of the base 102, but is still able to interact with the force distributing plate 103 to provide the force as described above.

Furthermore, as shown in FIG. 1A, the magnetic plate adapter 101 and the force distributing plate 103 each contain apertures 109. While such apertures 109 are shown as having a different size and arrangement in FIG. 1A, it should be understood that, in one aspect, the apertures 109 may be the same or different.

FIG. 1B illustrates the assembly of FIG. 1A in an assembled orientation Namely, the magnetic plate adaptor 101 is placed on a surface, and the base 102 is placed over the magnetic plate adaptor 101 in a manner that places the magnets 105 under the apron 107. Next, the lid 110 is placed over the base, and the force distributing plate 103 is aligned on the lid 110 such that the material, or magnets (not shown) are in magnetic contact with the magnets 105 of the magnetic plate adaptor 101 in order to provide a strong connection according to the present disclosure. As shown in FIG. 1B, the magnetic plate adapter 101 and force distributing plate 103 have a generally equal or lesser length and width than the base 102, such that no additional length or width is added to the assembly other than the standard size of the base 102 and lid 110.

In addition, FIG. 1C illustrates a cross section taken along the line C-C′ of FIG. 1B. as shown in FIG. 1C, the magnets 105 fit securely within apron 107 such that they are able to magnetically interact with force distributing plate 103 in order to provide the necessary downward force discussed above. Furthermore, as shown in FIG. 1C, the projections 112 have a highly consistent height clearance 154 (distance between most distal point of the distal end 116 and the well base 126). For instance, as discussed above, by selecting one or more vertically magnetized magnets 105 having a force according to the above ranges, the force distributing plate 103 is held in place with sufficient downward force to reduce warp and maintain the height clearance noted above. Namely, as shown, the force is able to be transferred via the magnet(s) 105 even with the base and lid disposed therebetween, and maintain proper force throughout testing as described above.

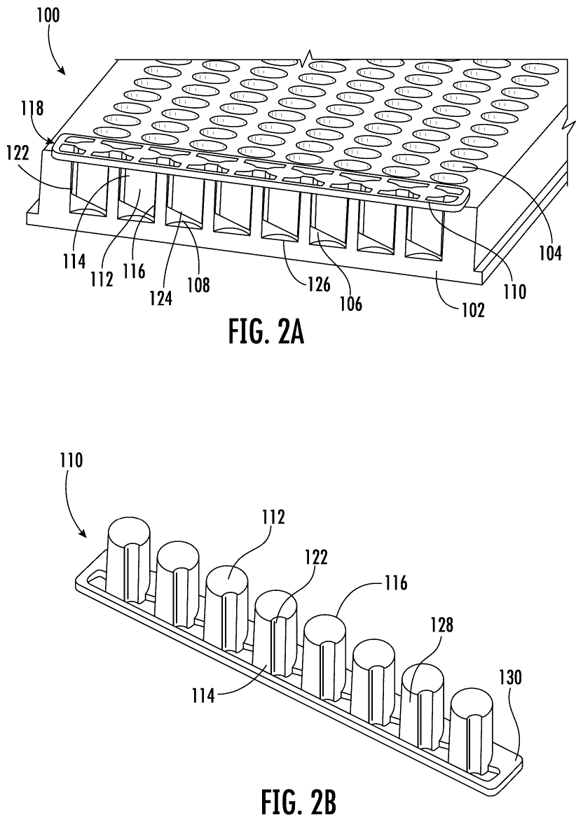

Referring to FIGS. 2A and 2B, an aspect of a base and lid for use in the assembly according to the present disclosure will be discussed in greater detail. Namely, as shown in FIG. 2A, a microtiter assembly 100 is illustrated having a base 102 having a plurality of wells 104 having at least one sidewall 106 (shown more clearly in FIG. 2B) which each define a cavity 108. Furthermore, FIG. 2A shows a lid 110 having a plurality of projections 112 which extend longitudinally from a proximal end 114 to a distal end 116 in a generally perpendicular manner from the lid 110. While shown more clearly in FIG. 2B, each projection 112 has a radial notch 122 which extends from the proximal end 114 to the distal end 116 of each projection 112. Furthermore, each projection 112 has a canted distal tip 124. Particularly, as discussed above, the canted distal tip 124 is angled according to the above discussed ranges relative to the plane of the well base 126 and/or lid 110.

As shown in FIG. 2A, a first row 118 of wells 102 have the respective cavities 108 at least partially occupied with the corresponding projection 112. While FIG. 2A illustrates an assembly having only a single row 118 of projections 112 attached to a lid 110, it should be understood from the above that the lid 110 can extend across the entire assembly and contain a corresponding number of projections 112 as the number of wells 104 in the respective base 102. Furthermore, in one aspect, each lid 110 may contain one or more strips containing projections 112, where each strip may be attached on one or more ends to a frame (shown more clearly in FIG. 4D below). For instance, in regards to FIG. 2A, eleven (11) additional strips with corresponding projections 112 can be attached to a frame such that each well 104 is occupied by a corresponding projection 112 to form a full ninety-six (96) well microtiter plate 100.

Referring next to FIG. 2B, a lid 110 is shown separately from the assembly 100 with the projections 112 extending from a proximal end 114 to a distal end 116, each having a radial notch 122 extending into the at least one sidewall 128 of each projection. As shown in FIGS. 2A and 2B, the projections 112 are connected to a support 130. Furthermore, while only a single row in shown in FIG. 2B, it should be understood that the support 130 may be used to connect multiple lids 110 to a frame (not shown in FIGS. 2A and 2B). However, it should also be understood that, in one aspect, the lid may be a single piece and may contain a number of projections corresponding to the respective base.

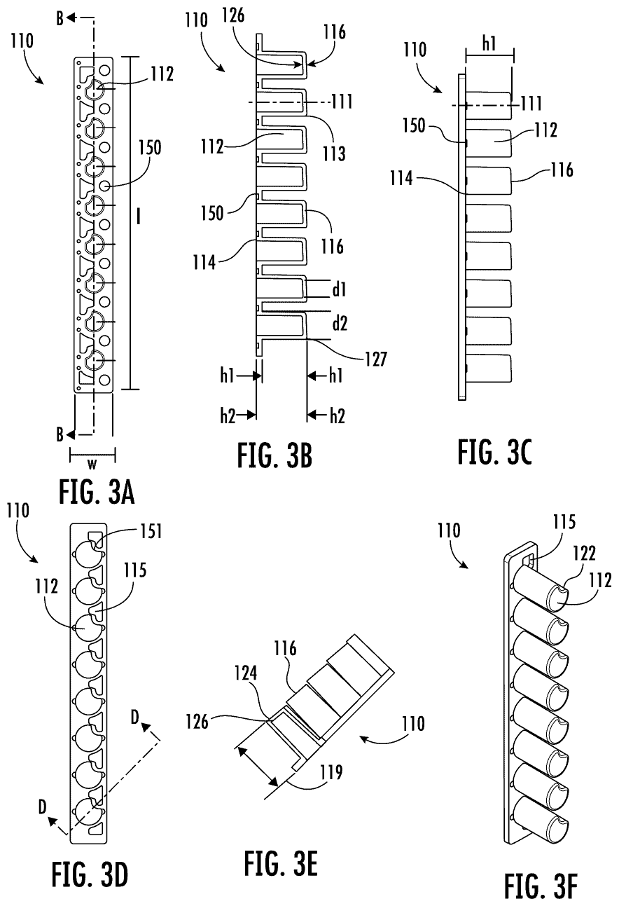

Similarly, FIGS. 3A-3F illustrate another aspect similar to FIGS. 2A and 2B, where lid 110 is in the form of a strip of eight projections 112. FIG. 2A shows a bottom-up view of the lid 110. As shown in FIG. 3A, the lid 100 in such an aspect can have a width w of about 12 mm or less, such as about 11 mm or less, such as about 10 mm or less, such as about 9 mm or less, such as about 5 mm or more, such as about 6 mm or more, such as about 7 mm or more, such as about 8 mm or more, or any ranges or values therebetween. For instance, in one aspect, the lid 110 has a width w from about 5 mm to about 12 mm, such as about 6 mm to about 11 mm, such as about 7 mm to about 9 mm, or any ranges or values therebetween.

Furthermore, as shown in FIG. 3A, which shows the apex 151 of the radial notch more clearly, the lid 110 can have a length/of about 100 mm or less, such as about 95 mm or less, such as about 90 mm or less, such as about 85 mm or less, such as about 80 mm or less, or such as about 60 mm or more, such as about 65 mm or more, such as about 70 mm or more, such as about 75 mm or more, or any ranges or values therebetween. For instance, in one aspect, the lid 110 has a length of about 60 mm to about 100 mm, such as about 65 mm to about 90 mm, such as about 70 mm to about 85 mm, or any ranges or values therebetween.

Similarly, referring to FIGS. 3B (which is a view along cross-section B-B of FIG. 2A) and 2C, in one aspect, the projections 112 have a height h1 from the stop tab 150 to a distal end 116 and a diameter d1, and the lid 110 has a height h2 from a stop tab receiving portion (not shown/shown more clearly in FIG. 4A) to a well base 126, and a diameter d2. Using these dimensions, the volume of the respective well 104 and the volume of the corresponding projection 112 can be determined to determine the fluid volume displaced by the lid 110. Furthermore, as shown in FIGS. 3B and 3C, the heights h1 and/or h2 can be measured from the center line 111 of each projection.

For instance, in one aspect, h1 is from about 15 mm or less, such as about 14 mm or less, such as about 13 mm or less, such as about 12 mm or less, such as about 11.5 mm or less, such as about 7 mm or greater, such as about 8 mm or greater, such as about 9 mm or greater, such as about 10 mm or greater, such as about 11 mm or greater, or any ranges or values therebetween. For instance, in one aspect, the lid 110 has a height h1 from about 7 mm to about 15 mm, such as about 9 mm to about 13 mm, such as about 10 mm to about 12 mm, or any ranges or volumes therebetween.

In addition, h2 can be about 17.5 mm or less, such as about 16.5 mm or less, such as about 15.5 mm or less, such as about 14.5 mm or less, such as about 13.5 mm or less, such as about 13 mm or less, such as about 8.5 mm or greater, such as about 9.5 mm or greater, such as about 10 mm or greater, such as about 10.5 mm or greater, such as about 11.5 mm or greater, such as about 12 mm or greater, or any ranges or values therebetween. For instance, in one aspect, the lid 110 has a height h2 from about 8.5 mm to about 17.5 mm, such as about 10 mm to about 16.5 mm, such as about 11.5 mm to about 14.5 mm, or any ranges or volumes therebetween. Furthermore, it should be understood that the ranges for h1 and/or h2 provided above, can be measured from the center line 111 or the most distal point of the distal tip 127 as shown in FIGS. 3B (most distal point 127) and 2C (center line 111)

Furthermore, in one aspect, d1 can be about 34.5 mm or less, such as about 29 mm or less, such as about 24 mm or less, such as about 19 mm or less, such as about 14 mm or less, such as about 9 mm or less, such as about 6.5 m or less, such as about 5 mm or less, such as about 4.9 mm or less, such as about 4.8 mm or less, such as about 4.5 mm or less, such as about 4 mm or less, such as about 3.5 mm or less, such as about 3 mm or less, such as about 2.5 mm or less, such as about 2 mm or less, such as about 1.5 mm or less, such as about 1 mm or more, such as about 2 mm or more, such as about 3 mm or more, such as about 4 mm or more, such as about 4.5 mm or more, such as about 5 mm or more, or any ranges or values therebetween. For instance, in one aspect, the inner diameter d1 can be from about 1 mm to about 34.5 mm, such as about 2 mm to about 19 mm, such as about 3 mm to about 9 mm, or any values or ranges therebetween.

Similarly, in one aspect, d2 can be about 35.5 mm or less, such as about 30 mm or less, such as about 25 mm or less, such as about 20 mm or less, such as about 15 mm or less, such as about 10 mm or less, such as about 7.5 m or less, such as about 5 mm or less, such as about 5 mm or more, such as about 5.5 mm or more, such as about 6 mm or more, such as about 6.3 mm or more, such as about 6.4 mm or more, such as about 6.45 mm or more. In one aspect, the outer diameter d2 can be from about 5 mm to about 35.5 mm, such as about 5.5 mm to about 25 mm, such as about 6 mm to about 20 mm, or any ranges or values therebetween. Furthermore, it should be understood that the ranges for d1 and/or d2 provided above, can be measured at the widest, or smallest diameter of the respective d1 and/or d2 in an aspect where the projections 112 are tapered.

In addition, as shown in FIG. 3B, in one aspect, the projections 112 can taper towards a distal end 116 in order to provide further reductions in overspilling without compromising on average wall clearance and evaporation reduction. Thus, in one aspect, one or more projections 112 has a center line 111 which is generally perpendicular to lid 110, and each projection has a taper 113 having an angle relative to the center line 111 of about 2° or less, such as about 1.75° or less, such as about 1.5° or less, such as about 1.25° or less, such as about 1° or less, such as about 0.75° or less, or about 0.25° or greater, such as about 0.4° or greater, such as about 0.65° or greater, or any ranges or values therebetween. For instance, in one aspect, one or more (or each) projections has a taper from about 0.25° to about 2°, such as about 0.35° to about 1.75°, such as about 0.55° to about 1.5°, or any ranges or values therebetween.

Referring next to FIGS. 3D and 3E, where FIG. 3D is a top-down view of FIG. 3A, and FIG. 3E is a view of the cross-section taken along line D-D. Apertures 115 are illustrated adjacent to each radial notch 122. Nonetheless, as shown most clearly in FIG. 3E, the canted distal tip 124 has an angle relative to the plane 119 of the lid 100, which, as shown, may be generally planar. Furthermore, while the plane 119 of the lid 110 and the well base 126 was referred to above as being in parallel planes, as shown in FIG. 3E, it should be understood that, in one aspect, the well base 126 is canted at the same, or similar, angle to the canted distal tip 124. In such a manner, the well base 126 has a consistent thickness (not shown) across its entire cross section. However, as noted above, in one aspect, the well base 126 can be in a plane generally parallel to the plane 119 of the lid, such that the well base 126 has a thickness than increases towards the most distal point of the canted distal tip 124.

Finally, FIG. 3F illustrates a perspective view of the lid 110 of FIG. 3A-3F. as shown, in an aspect having an aperture 115, the aperture 115 may be adjacent to the radial notch 122.

Moreover, as shown in FIG. 4A, in one aspect, the lid 110 can be a single piece having a number of projections 112 equal to a corresponding base 102 (not pictured). Each projection 112 has a radial notch 122 that extends into the at least one sidewall 128 of each projection 112. In addition, the lid 110 contains a frame 132 having a planar portion 134 and a lip 136. The lip 136 can contain one or more angled corners 138, or may have one or more square corners 140. In one aspect, as shown in FIGS. 4A and 4B, two of the corners may be angled in order to provide a reversible locking function to the lid 110 by providing a tighter fit and seal to maintain the lid 110 on the base (not shown) and to further assist with evaporation.

Furthermore, the lip 136 may also have a length L3 that maintains SBS compatibility, but that extends further along the base 102 then prior lids. For instance, the length L3 may be from about 10 mm or more, such as about 10.25 mm or more, such as about 10.5 mm or more, such as about 10.75 mm or more, such as about 11 mm or more, such as about 11.25 mm or more, such as about 11.5 mm or more, up to about 12 mm or less, such as about 11.75 mm or less, such as about 11.7 mm or less, such as about 10 mm to about 12 mm, or about 10.25 mm to about 11.75 mm, such as about 10.5 mm to about 11. 7 mm, or any ranges or values therebetween. Namely, the present disclosure has found that lips 136 having lengths L3 within the above discussed ranges may further aid in reducing evaporation, which is of heightened importance when using reduced sample sizes

Further, FIG. 4B illustrates a base 102 that cooperates with the lid 110 of FIG. 4A. As shown, the base 102 contains a support 142 having a skirt 141 that contains a plurality of chambers or wells 104. In the illustrated aspect, the base 102 also contains angled corners 138, which releasably associate with the angled corners 138 of the lid of FIG. 4A. As discussed above, the lid 110 of FIG. 4A may cooperate with each well 104 of FIG. 4B to occupy a portion of the cavity 108 formed by well sidewalls 108. While not shown, it should be understood that when a sample is present, the lid 110 may displace some or all of a gas located between the lid 108 and a sample (not shown), and, in one aspect, may evacuate all of a gaseous headspace contained in each well 104 such that each projection 112 contacts a sample (not shown) contained in the respective well 104.

In addition, FIG. 4C illustrates an assembly 100 according to the present disclosure, where the lid 110 (shown more clearly in FIG. 4A as discussed above) is disposed onto the base 102 (shown more clearly in FIG. 4B above), such that each projection 112 is in reversible association with the respective wells 104 (shown more clearly in FIG. 4B).

FIG. 4D illustrates another aspect of an assembly 100 according to the present disclosure, where the lid 110 is shown in two parts, frame 152 and cover 153. Namely, as shown rows of strips 156 containing projections 112 are releasably affixed to the frame 152. In such a manner, the frame 152 may be formatted to include the correct number of strips 156 for the number of wells 104 contained in the respective base 102. Furthermore, in such an aspect, the cover 153 and/or frame 152 may be formed of any of the substrate materials discussed above, or alternatively, the frame 152 may be formed from a rigid material, such as a metal, which can be aluminum in one aspect. Nonetheless, in one such aspect, the cover 153 may be clear or frosted.

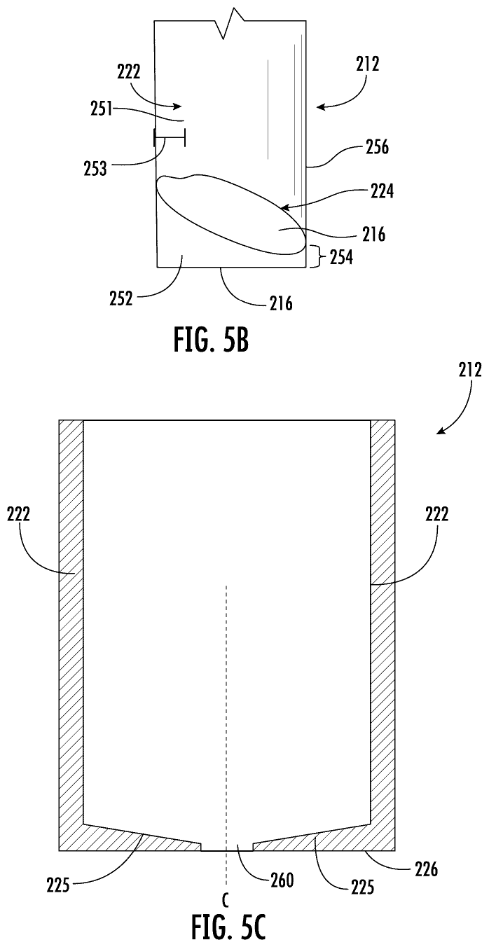

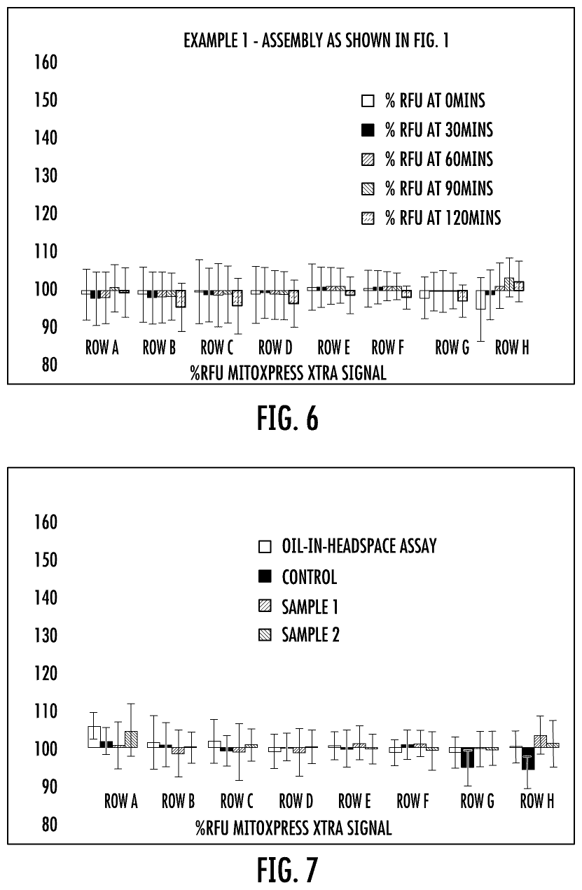

Referring next to FIGS. 5A and 5B, a microtiter plate assembly 200 is shown having a lid 210 having a planar portion 234 and a lip 236. As discussed above, in one aspect, the frame (132 above) may be integral to the lid 210. Furthermore, the lid 210 has a plurality of projections 212 which extend from a proximal end 214 adjacent to the cover plate 210 to a distal end 216 having a canted distal tip 224. As shown in FIG. 5A, the projections 212 may generally be hollow, and have an external sidewall 246 that contacts the sample, and an internal sidewall 248 that is located on an internal potion of each projection. However, even if the projection is hollow, it should be understood that the radial notch 222 does not penetrate through the external sidewall 246 into the internal sidewall 248. In one aspect, for instance, the projections 212 are hollow as they are formed by injection molding, however, it should be understood that any method known in the art may be used, such as milling.

In addition, FIG. 5A shows more clearly the stop tabs 250. As discussed above, the stop tabs 250 may be used in conjunction with the radial notch 222 and canted distal tip 224 to provide highly accurate height and wall clearances, which will be discussed in greater detail in regards to FIG. 5B. Namely, the stop tabs 250 contact a portion of the base 202 to dispose the projections 212 at specific heights so as to maintain the necessary height and wall clearances (shown more clearly in FIG. 5B). In one aspect, the stop tab 250 may contact a continuous portion of the base 202, or alternatively, may contact a stop tab receiving portion 258.

Nonetheless, as shown, the microtiter plate assembly 200 also includes a plurality of wells 244 which are configured to releasably contain the projections 212 in a reversible manner.

Furthermore, as shown more clearly in FIG. 5A, the projections 212 have a height h1 from the stop tab 250 to a distal end 216 and a diameter d1, and the lid 210 has a height h2 from the stop tab 250 height to a well base 226, and a diameter d2. Using these dimensions, the volume of the respective well 204 and the volume of the corresponding projection 212 can be determined to determine the fluid volume displaced by the lid 210.

FIG. 5B contains a close-up depiction of the distal end 216 of a projection 212 of FIG. 5A. Namely, as shown in FIG. 5B, the distal end 216 of the projection 212 has a canted distal tip 224 relative to the well base 226 (e.g., the lower surface of the interior of the well cavity). Furthermore, FIG. 5B more clearly illustrates the sample 252, height clearance 254, wall clearance 256, radial notch 222, apex 251, and radial notch clearance 253 as discussed above. Namely, as shown, the small, but accurate height clearance 254 (e.g., the distance between the most distal portion of the projection 212/portion of the projection 212 closest to the well base 226) is close to the well base 226 to allow sensitive measurements without touching the well base 226 which would interrupt cell growth and seeding. Furthermore, as shown, the wall clearance 256 is very small so as to enable the projection 212 to be removed from the well 244 while still guiding any air bubbles to the radial notch 222.

Furthermore, FIG. 5B shows that the radial notch 222 is located adjacent to the highest portion of the canted distal tip 224 (e.g., the portion of the distal end 216 having the shortest distance from the proximal end 224 or the portion of the canted distal tip 224 furthest from the well base 226). In such a manner, bubbles may be guided by the angle of the canted distal tip 224 to the radial notch 222 so that bubbles may be efficiently evacuated from the sample 252. As shown in FIG. 4B, the sample 252 has a height h that is slightly higher than the sum of the height of the canted portion of the distal tip 224 and the height clearance 254. In such a manner, the sample 252 may extend into the radial notch 222 such that overspilling is prevented but the headspace is fully eliminated.

In addition, FIG. 5C illustrates an aspect of the present disclosure where the projection 212 includes two radial notches 222 and two angled surfaces 225 on the canted distal tip 224, that have a most distal end adjacent to center line C and a highest end adjacent to the respective notch 222. Furthermore, as shown, the projection 212 contains a spotting surface 260, which is generally perpendicular to the well base 226.

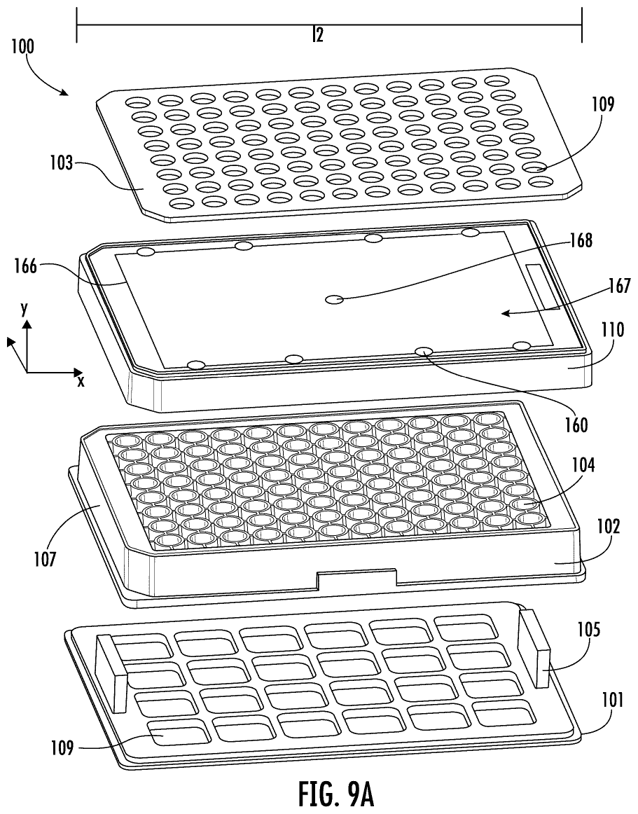

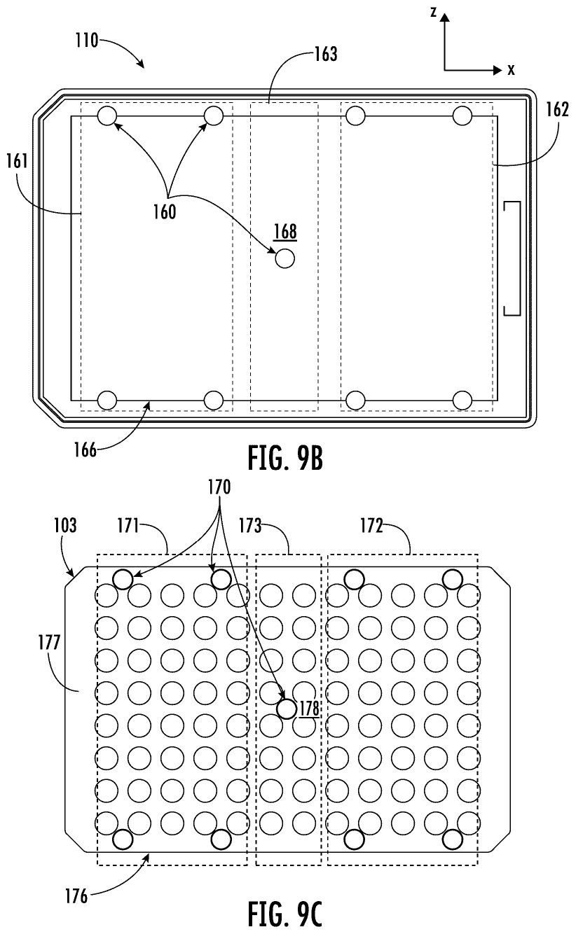

Meanwhile, FIGS. 9A-9C illustrate additional aspects of a lid and force distributing plate for use in the assembly according to the present disclosure. Namely, as shown in FIG. 9A, a microtiter plate assembly 100 is illustrated having a base 102 having a plurality of wells 104. Furthermore, FIG. 9A shows a lid 110 having a plurality of projections (not shown) which extend longitudinally from the lid 110 as described above with respect to FIG. 1A. The lid 110 is shown from a top view in FIG. 9B. In particular, the lid 110 can include a plurality of spacers 160 disposed on its exterior surface 167. The spacers 160 can be located about a perimeter 166 of the lid 110, and/or at the center 168 of the lid 110, or in any suitable location. The present inventors have found that the use of such spacers 160 can prevent warping of the lid 110 by increasing downward pressure on the base 102 via the lid 110 to provide more accurate test results and achieve sufficient sample volume consistency across the base 102, leading to more consistent oxygen depletion rates across a full base 102.

As shown in FIG. 9B, multiple spacers 160 can be present within a first outer region 161 of the lid 110, multiple spacers 160 can be present within a second outer region 162 of the lid 110, and a single spacer 160 can be located within a central region 163 of the lid 110. However, it is to be understood that additional spacers 160 can be located within any of the regions 160, 161, or 162 to achieve reduced warpage of the lid 110. Further, the spacers 160 can be injection molded during the formation of the lid 110 and can be formed from the same material as the rest of the lid 110.

Moreover, it should be understood that the spacers 160 can have varying thicknesses in the y-direction in order to provide uniformity and reduced warpage of the lid 110. However, in other embodiments, the spacers 160 can each have the same thickness. For instance, in some embodiments, the spacers 160 can each have a thickness ranging from about 100 micrometers to about 500 micrometers, such as from about 125 micrometers to about 475 micrometers, such as from about 150 micrometers to about 450 micrometers.

In one particular embodiment, the spacers 160 in the central region 163 can have a thickness that is greater than the thickness of the spacer 160 in the first outer region 161 and the second outer region 162 to provide increased downward pressure at the center of the lid 110 where more warpage can occur. In such an embodiment, the spacers 160 in the central region 163 can have a thickness ranging from about 175 micrometers to about 500 micrometers, such as from about 200 micrometers to about 475 micrometers, such as from about 225 micrometers to about 450 micrometers, while the spacers 160 in the first outer region 161 and the second outer region 162 can have a thickness ranging from about 100 micrometers to about 250 micrometers, such as from about 125 micrometers to about 225 micrometers, such as from about 150 micrometers to about 200 micrometers.

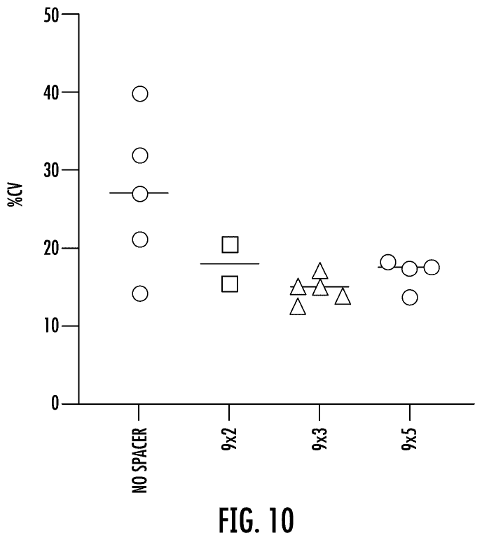

As shown in FIG. 10, a comparison of the coefficient of variation of oxygen depletion measurements in the wells of a lid 110 of the microtiter plate assembly 100 of FIGS. 9A and 9B utilizing spacers 160 having various thicknesses compared to a lid 110 with no spacers shows that the coefficient of variation (% CV) is reduced when spacers 160 are utilized compared to when no spacers are utilized.

Turning now to FIG. 9C, in addition to or alternatively to the lid 110, the force distributing plate 103 can also include a plurality of spacers 170 disposed on an interior surface 177 of the force distributing plate 103. The spacers 170 can be located about a perimeter 176 of force distributing plate 103, and/or at the center 178 of the plate 103, or in any suitable location. The present inventors have found that the use of such spacers 170 can prevent warping of the lid 110 by increasing downward pressure on the base 102 via the plate 103 to provide more accurate test results. As shown in FIG. 9C, multiple spacers 170 can be present within a first outer region 171 of the plate 103, multiple spacers 170 can be present within a second outer region 172 of the plate 103, and a single spacer 170 can be located within a central region 173 of the plate 103. However, it is to be understood that additional spacers 170 can be located within any of the regions 170, 171, or 172 to achieve reduced warpage of the lid 110 via the plate 103 and achieve sufficient sample volume consistency across the base 102, leading to more consistent oxygen depletion rates across a full base 102.

Moreover, it should be understood that the spacers 170 can have varying thicknesses in the y-direction in order to provide uniformity and reduced warpage of the lid 110 via the plate 103. However, in other embodiments, the spacers 170 can each have the same thickness. For instance, in some embodiments, the spacers 170 can each have a thickness ranging from about 100 micrometers to about 500 micrometers, such as from about 125 micrometers to about 475 micrometers, such as from about 150 micrometers to about 450 micrometers. In one particular embodiment, the spacers 170 in the central region 173 can have a thickness that is greater than the thickness of the spacers 170 in the first outer region 161 and the second outer region 172 to provide increased downward pressure at the center of the lid 110 where more warpage can occur. In such an embodiment, the spacers 160 in the central region can have a thickness ranging from about 175 micrometers to about 500 micrometers, such as from about 200 micrometers to about 475 micrometers, such as from about 225 micrometers to about 450 micrometers, while the spacers 170 in the first outer region 171 and the second outer region 172 can have a thickness ranging from about 100 micrometers to about 250 micrometers, such as from about 125 micrometers to about 225 micrometers, such as from about 150 micrometers to about 200 micrometers.

Aspects of the present disclosure may be further understood according to the following nonlimiting examples.

### Example 1

A microtiter plate assembly according to FIGS. 1A-1C was formed having a magnetic plate adapter, having twenty-four (24) apertures and two magnets oriented perpendicular to the magnetic plate adapter and being magnetized in the horizontal direction. As shown in FIG. 1A, the magnets were disposed near the center point of opposed ends of the magnetic plate adapter, spaced apart in the longitudinal direction. Each magnet had a magnetic force of about 4 kgf for a total magnetic force of about 8.75 kgf. A base and lid as illustrated in FIG. 1A were provided over the magnetic plate adapter, and a force distribution plate formed of coated steel with black zinc oxide having ninety-six (96) circular apertures was placed over the lid.

Stability of the lid, microplate and adapter assembly was evaluated by comparing average MitoXpress Xtra signal from outside rows to inside rows of wells (e.g., where outside rows are rows on an outer edge of the assembly such as the first and last row, and inside rows are contained between opposed outside rows). The MitoXpress Xtra reagent is dissolved in the sample volume, so that an increased signal would be an indication of an increased sample volume. Acquired data was assessed by calculating the average fluorescence signal and standard deviation from each row of wells (e.g., A1 to A12, B2 to B12 etc.) on the 96 well plate. That average signal is then expressed as a % of the average signal of the middle Rows (C, D, E & F). This made it possible to compare differences in outside edges to inside/center wells/samples of the assembly.

Signal was measured at 0, 30, 60, 90, and 120 minute time points, in order to evaluate changes in stability (change in well volumes related to movement of the pillar/lid) over time.

Signal change less than +/−10% indicates very stable signal/assembly.

As shown in the following examples, without a downward pressure device, signal change in outer rows could exceed 25-30%, due to lid corners moving or raising up during the assay. As the lid is assembled as one solid piece, this also impacted neighboring wells, resulting in consistent measurements from only a few wells in the center of the plate.

Throughout the example, the temperature was maintained at 37° C., and fluorescence was determined using a BMG Clariostar and using a standard MitoXpress Xtra assay protocol.

After the assembly was assembled as shown in FIG. 1B, a warp test was conducted as defined above, and the RFUs were measured over time. As shown in FIG. 6, the assembly of the present disclosure exhibited excellent warp even at 120 minutes.

### Example 2

Example 2 was conducted in a similar method as Example 1. In addition, an oil-in-headspace sample, and a sample having 7 permanently affixed screws holding the assembly together were tested along with the two samples using the warp test as defined above for a run time of 90 minutes. Namely, as discussed above, increase signal over time indicates upward movement of the lid and therefore, increased sample volume, which is caused by warping of the lid. As shown in FIG. 7, the assemblies according to present disclosure exhibited less than 10% warping.

### Example 3

The assembly of Example 1 was tested for sensitivity against a standard oil-in-headspace assay. Seeding densities of 10,000, 35,000, and 70,000 were tested for both the sample assay assembly and the oil-in-headspace assay using a MitoXpress Xtra and CLARIOstar plate reader as described above to determine sensitivity to rate of oxygen consumption of C2C12 cells. The testing yielded the following results:

TABLE 1

| Sample 1 | | Oil-in-headspace |
|---|---|---|
| 10K C2C12 | 8.4 μs/h, | Undetectable |
| | % CV 12.9 | |
| 35K C2C12 | 47.2 μs/h, | Undetectable |
| | % CV 18.4 | |
| 70K C2C12 | N/A | Detectable, |
| | | over-confluent |
| | | ~4 μs/h, |
| | | % CV 15-20 |

As shown in Table 1, the assembly according to the present disclosure was able to detect the rate of oxygen consumption below or at confluence in 30 to 45 minutes.

### Example 4

An assembly according to Example 1 was formed, where the plate adapter and force distributing plate were formed from steel. A Starrett Granite Surface Plate with an electronic digital indicator (2720-0M) was used to measure point of deflection in outer row(s) under stress testing according to the following

Assembling the microtiter plate assembly by placing well/microplate assembly on a plate adapter that contains one or more magnets as discussed above and placing a steel force distributing plate over the lid of the well/microplate assembly. After assembly at room temperature, the microtiter plate assembly was placed in a heated environment of 37° C., and left for two hours. After the two hours of heating, the assembly was removed from heat, disassembled, and allowed to cool to room temperature.

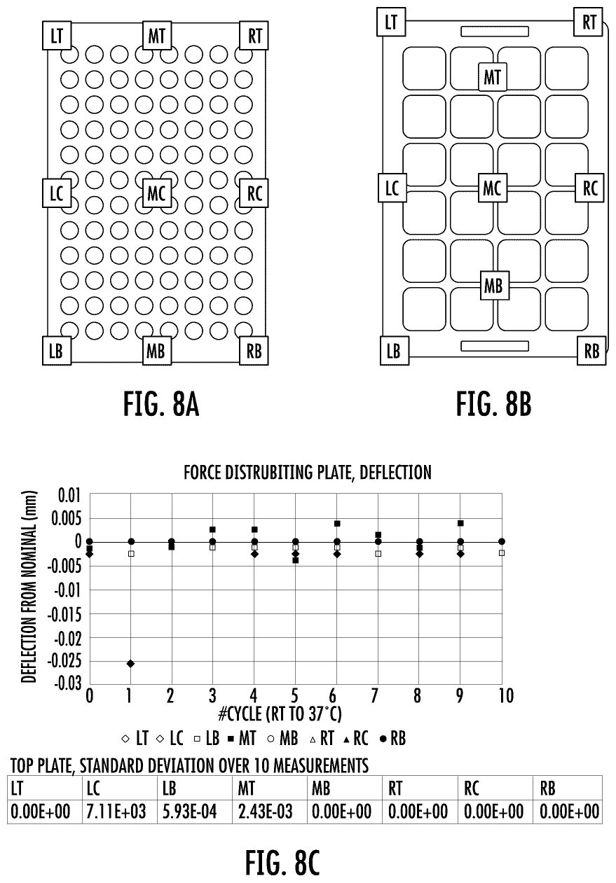

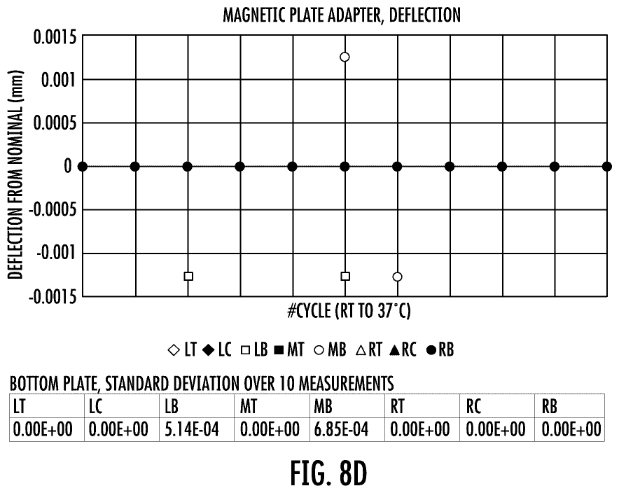

After cooling, the plate adapter and force distributing plate were measured to determine flatness at the nine points indicated in FIGS. 8A and 8B, by measuring the distance between the bottom of the respective plate and the measurement surface at each of the locations. The flatness measurements were also recorded under deflection, where the deflection occurred at a point opposite the measurement location (e.g. if measuring at LT, deflection at RB). The heating, cooling, and measurements were then repeated 9 more times, for a total of 10 cycles. The average deflection measurements are shown in FIGS. 8D and 8D. As shown, even utilizing the extremely sensitive Starrett system, the plate adapter and force distributing plate exhibited excellent flatness over the stress testing cycles.

These and other modifications and variations to the present disclosure may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. A microtiter plate assembly comprising:
a base comprising a plurality of wells in at least one outer row and at least one inner row;
a lid comprising a plurality of projections corresponding with the plurality of wells;
a magnetic plate adapter; and
a force distributing plate,
wherein at least a portion of the force distributing plate, the magnetic plate adaptor, or both the magnetic plate adapter and force distributing plate is formed from a magnetic or paramagnetic material, or has a magnetic or paramagnetic material disposed thereon, or has a magnetic material attached thereto;
wherein the at least one magnet provides a force sufficient to reduce warping of the lid; and
wherein the assembly exhibits a percent warp, defined as a percent change in signal of an average fluorescence signal of the at least one outer row to an average fluorescence signal of the at least one inner row, of about 30% or less.

2. A microtiter plate assembly comprising:
a base comprising a plurality of wells spaced apart such that each well is adjacent to a second well; and
a lid comprising a plurality of projections spaced apart corresponding with the array of the plurality of wells such that each projection is adjacent to a second projection;
a magnetic plate adapter having at least one magnet; and
a force distributing plate,
wherein at least a portion of the force distributing plate, the magnetic plate adaptor, or both the magnetic plate adapter and force distributing plate is formed from a magnetic or paramagnetic material, or has a magnetic or paramagnetic material disposed thereon, or has a magnetic material attached thereto;
wherein the plurality of projections extend from a proximal end adjacent to the lid to a distal end; and
wherein a height clearance is defined between the distal end of each projection and a well base of the respective well, and wherein a percent variation of the height clearance between the projection and the well and the adjacent second projection and second well is about 10% or less.

3. The microtiter plate assembly as defined in claim 1 or 2, wherein the force distributing plate, the magnetic plate adaptor, or both the magnetic plate adapter and force distributing plate provides at least about 4 kilograms*force (kgf) to the lid.

4. The microtiter plate assembly as defined in any one of the preceding claims, wherein the assembly exhibits a percent warp of about 15% or less.

5. The microtiter plate assembly as defined in any one of the preceding claims, wherein at least one of the magnetic plate adapter and the force distributing plate has a longitudinal length that is equal to or less than a longitudinal length of the base.

6. The microtiter plate assembly as defined in any one of the preceding claims, wherein the base contains an apron, wherein the apron is configured to releasably contain the at least one magnet.

7. The microtiter plate assembly as defined in any one of the preceding claims, wherein the microtiter plate assembly is configured to be compatible with a plate reader and/or is Society for Biomolecular Screening (SBS) compliant.

8. The microtiter plate assembly as defined in any one of the preceding claims, wherein the at least one magnet is vertically magnetized.

9. The microtiter plate assembly as defined in any one of the preceding claims, wherein at least one of the magnetic plate adapter and the force distributing plate has a thickness of about 12 mm or less.

10. The microtiter plate assembly as defined in any one of the preceding claims, wherein at least one of the magnetic plate adapter and the force distributing plate has a weight of about 600 g or less.

11. The microtiter plate assembly as defined in any one of the preceding claims, wherein at least one of the magnetic plate adapter and the force distributing plate contains one or more apertures.

12. The microtiter plate assembly as defined in any one of the preceding claims, wherein the assembly includes 96 wells and 96 corresponding projections.

13. The microtiter plate assembly as defined in any one of the preceding claims, wherein at least one of the magnetic plate adapter and the force distributing plate is formed of aluminum, steel, combinations thereof, or alloys or derivatives thereof.

14. The microtiter plate assembly as defined in any one of the preceding claims, wherein each projection comprises a radial notching having a radial cross section of about 0.8 mm² or greater.

15. The microtiter plate assembly as defined in any one of the preceding claims, wherein the distal end of each projection is canted at an angle of about 5° or more relative to a plane of the lid.

16. The microtiter plate assembly as defined in any one of the preceding claims, wherein at least a portion of the assembly is formed from an acrylate polymer, or wherein at least a portion of the assembly is coated with a polymer having an oxygen transmission rate of about 15 cm³/m²/24 hours at 23° C. and 0% relative humidity.

17. 17. The microtiter plate assembly as defined in any one of the preceding claims, wherein at least one of the lid and the force distributing plate includes a plurality of spacers.

18. A method of measuring an oxygen consumption rate utilizing the assembly of any one of the preceding claims comprising:
placing a sample and an oxygen-sensitive phosphorescent probe into one of the plurality of wells,
contacting the sample with the corresponding projection, and
measuring the oxygen consumption rate with a fluorescence plate reader.

19. The method as defined in claim 18, wherein the sample has a seeding density of less than 70,000 cells, and wherein the measuring is conducted in 45 minutes or less.

20. The method as defined in claim 18 or 19, wherein the seeding density is conducted at or below confluence of a selected cell type.

21. The method as defined in any one of claims 18 to 20, wherein the sample volume is 100 μL or less, preferably wherein the sample volume is 70 μL or less.
